(19) United States
(12) Patent Application Publication
(10) Pub. No.: US 2024/0182420 A1
(43) Pub. Date: Jun. 6, 2024

(54) MICROBIOCIDAL ISONICOTINIC AMIDE DERIVATIVES

(71) Applicant: SYNGENTA CROP PROTECTION AG, Basel (CH)

(72) Inventors: Thomas James HOFFMAN, Stein (CH); Matthias WEISS, Stein (CH); Myriem EL QACEMI, Stein (CH); Thomas PITTERNA, Stein (CH); Clemens LAMBERTH, Stein (CH); Vlad PASCANU, Stein (CH); Andrew EDMUNDS, Stein (CH); Ramya RAJAN, Corlim, Ilhas (IN); Nicolas GERMAIN, Stein (CH)

(73) Assignee: SYNGENTA CROP PROTECTION AG, Basel (CH)

(21) Appl. No.: 18/552,704

(22) PCT Filed: Mar. 25, 2022
(86) PCT No.: PCT/EP2022/057880

§ 371 Date: Sep. 27, 2023

(30) Foreign Application Priority Data
Mar. 27, 2021 (IN) 202111013707
Jun. 8, 2021 (EP) 21178372.5
Nov. 11, 2021 (EP) 21207851.3

(51) Int. Cl. C07D 213/81; A01N 43/40; A01P 3/00

(52) U.S. Cl. CPC C07D 213/81; A01N 43/40; A01P 3/00

(57) ABSTRACT

Compounds of Formula (I) wherein the substituents are as defined in claim 1, useful as pesticides, especially as fungicides.

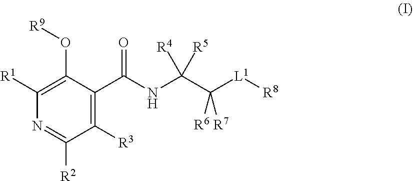

The present invention relates to isonicotinic amide derivatives, e.g., as active ingredients, which have microbiocidal activity, in particular, fungicidal activity. The invention also relates to agrochemical compositions which comprise at least one of the isonicotinic amide derivatives, to processes of preparation of these compounds and to uses of the isonicotinic amide derivatives or compositions in agriculture or horticulture for controlling or preventing infestation of plants, harvested food crops, seeds or non-living materials by phytopathogenic microorganisms, preferably fungi.

WO 2020/109391 discloses the use of pyridazine (thio)amide derivatives for controlling phytopathogenic microorganisms. WO 2021/224220 discloses the use of pyridine (thio)amides a fungicidal compounds. JP 2001 294563 discloses salicylic acid amides having bactericidal and herbicidal activity. WO 2017/012966 discloses hydrazides useful for increasing stress tolerance in plants with respect to abiotic stree.

According to the present invention, there is provided a compound of Formula (I):

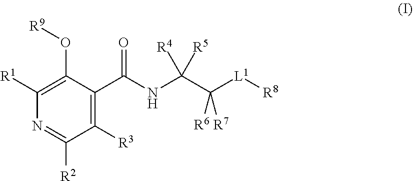

wherein:
L¹ is —O— or —CH₂— or a bond;
R¹ is selected from hydrogen, halogen, cyano, C₁-C₃-alkyl or C₁-C₃-alkoxy;
R² is selected from hydrogen, cyano, mercaptyl, halogen, C₁-C₃-alkyl, C₂-C₄-alkenyl, such as C₂-C₃-alkenyl, C₂-C₃-alkynyl, C₁-C₃-alkoxy, C₁-C₃-fluoroalkyl, C₁-C₃-fluoroalkoxy, C₃-C₄-cycloalkyl or C₁-C₃-alkylsulfonyl;
R³ is selected from hydrogen, halogen, cyano, methyl, C₁-C₄-alkyl, C₁-C₃-fluoroalkyl, C₂-C₄-alkenyl, C₂-C₄-alkynyl, C₁-C₄-alkoxy, C₂-C₄-alkenyloxy, C₂-C₅-alkynyloxy, C₁-C₃-fluoroalkoxy, C₃-C₄-cycloalkyl; or
R³ is phenoxy optionally substituted with 1 or 2 substituents independently selected from halogen, cyano, methyl, ethyl, difluoromethyl, trifluoromethyl, methoxy, ethoxy, difluoromethoxy, trifluoromethoxy, cyclopropyl,
R⁴ and R⁵ are independently selected from hydrogen, cyano or methyl; or
R⁴ and R⁵, together with the carbon atom to which they are attached, form a cyclopropyl or cyclobutyl group;
R⁶ and R⁷ are independently selected from hydrogen, fluoro, chloro, methyl, trifluoromethyl, hydroxy, methoxy, methoxymethyl, difluoromethoxy, mercaptyl, methylsulfanyl; or
R⁶ and R⁷, together with the carbon atom to which they are attached, form an oxo, cyclopropyl, or cyclobutyl group, or a 4- to 6-membered monocyclic heterocycle group which comprises 1 or 2 heteroatoms individually selected from N, O and S; or
R⁴ and R⁶, together with the carbon atom to which they are attached, form a C₃-C₄-cycloalkyl or a 4- to 6-membered monocyclic heterocycle group which comprises 1 or 2 heteroatoms individually selected from N, O and S, and R⁵ and R⁷ are independently hydrogen or halogen;
R⁸ is phenyl optionally substituted with:
a single substituent selected from C₁-C₄-alkyl, C₁-C₂-haloalkyl, C₁-C₃-alkoxy, C₁-C₃-alkylsulfanyl, C₁-C₃-alkylsulfonyl, C₁-C₂-haloalkoxy, C₂-C₃-alkenyl, C₂-C₃-haloalkenyl, C₂-C₃-alkynyl, C₃-C₄-cycloalkyl, C₃-C₄-cycloalkyloxy or —NH(COC₁-C₃-alkyl), or
1, 2 or 3 substituents that may be the same or different, independently selected from hydroxyl, halogen, mercapto, amino, cyano, methyl, ethyl, propyl, isopropyl, difluoromethyl, trifluoromethyl, methoxy, ethoxy, propyloxy, isopropyloxy, methylsulfanyl, methylsulfonyl, difluoromethoxy, trifluoromethoxy, cyclopropyl, cyclobutyl, cyclopropyloxy, —NH(COCH₃); and
R⁹ is phenyl optionally substituted with:
a single substituent selected from C₁-C₄-alkyl, C₁-C₂-haloalkyl, C₁-C₄-alkoxy, C₁-C₃-alkylsulfanyl, C₁-C₃-alkylsulfonyl, C₁-C₂-haloalkoxy, C₂-C₃-alkenyl, C₂-C₃-haloalkenyl, C₂-C₃-alkynyl, C₃-C₄-cycloalkyl, C₃-C₄-cycloalkyloxy; or
1, 2 or 3 substituents which may be the same or different, independently selected from hydroxyl, halogen, mercapto, amino, cyano, methyl, ethyl, propyl, isopropyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, cyclopropyloxy, —NH(COCH₃); or
or a salt or an N-oxide thereof.

Surprisingly, it has been found that the novel compounds of Formula (I) have, for practical purposes, a very advantageous level of biological activity for protecting plants against diseases that are caused by fungi.

According to a second aspect of the invention, there is provided an agrochemical composition comprising a fungicidally effective amount of a compound of Formula (I). Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of Formula (I), ora composition comprising this compound as active ingredient, is applied to the plants, to parts thereof or the locus thereof.

According to a fourth aspect of the invention, there is provided the use of a compound of Formula (I) as a fungicide. According to this particular aspect of the invention, the use may exclude methods for the treatment of the human or animal body by surgery or therapy.

As used herein, the term “hydroxyl” or “hydroxy” means an —OH group.

As used herein, the term “mercapto” means an —SH group.

As used herein, the term “cyano” means a —CN group.

As used herein, amino means an —NH₂ group.

As used herein, nitro means an —NO₂ group.

As used herein, oxo means an ═O group (eg, as in a carbonyl (C═O) group).

As used herein, the term “halogen” or “halo” refers to fluorine (fluoro), chlorine (chloro), bromine (bromo) or iodine (iodo), preferably fluorine, chlorine or bromine.

As used herein, the term “C₁₋₄alkyl” refers to a straight or branched hydrocarbon chain radical consisting solely of carbon and hydrogen atoms, containing no unsaturation, having from one to four carbon atoms, and which is attached to the rest of the molecule by a single bond. C₁₋₃alkyl should be construed accordingly. Examples of C₁₋₄alkyl include, but are not limited to, methyl, ethyl, iso-propyl.

As used herein, the term “C₂₋₃alkenyl” refers to a straight or branched hydrocarbon chain radical group consisting solely of carbon and hydrogen atoms, containing at least one double bond that may be of either the (E) or (Z) configuration, having two or three carbon atoms, which is attached to the rest of the molecule by a single bond. Examples of C₂₋₃alkenyl include, but are not limited to, prop-1-enyl, allyl (prop-2-enyl).

As used herein, the term “C₂₋₃haloalkenyl” refers to a C₂₋₃alkenyl group as defined above substituted by one or more of the same or different halogen atoms.

As used herein, the term “C₂₋₃alkynyl” refers to a straight or branched hydrocarbon chain radical group consisting solely of carbon and hydrogen atoms, containing at least one triple bond, having from two or three carbon atoms, and which is attached to the rest of the molecule by a single bond. Examples of C₂₋₃alkynyl include, but are not limited to, prop-1-ynyl and propargyl (prop-2-ynyl).

As used herein, the term “C₁₋₃alkoxy” refers to a radical of the formula RₐO— where Rₐ is a C₁₋₃alkyl radical as generally defined above. Examples of C₁₋₃alkoxy include, but are not limited to, methoxy, ethoxy, iso-propoxy.

As used herein, the term “C₁₋₄haloalkyl” refers to a C₁₋₄alkyl radical as generally defined above substituted by one or more of the same or different halogen atoms. Examples of C₁₋₄haloalkyl include, but are not limited to fluoromethyl, fluoroethyl, difluoromethyl, trifluoromethyl, and 2,2,2-trifluoroethyl.

As used herein, the term “C₁₋₃fluoroalkyl” refers to a C₁₋₃alkyl radical as generally defined above substituted by one or more fluorine atoms. Examples of C₁₋₃fluoroalkyl include, but are not limited to difluoromethyl and trifluoromethyl.

As used herein, the term “C₁₋₃fluoroalkoxy” refers to a C₁₋₃alkoxy radical as generally defined above substituted by one or more fluorine atoms. Examples of C₁₋₃fluoroalkoxy include, but are not limited to trifluoromethoxy.

As used herein, the term “C₃₋₄cycloalkyl” refers to a stable, monocyclic ring radical which is saturated and contains 3 or 4 carbon atoms.

As used herein, the term “C₁₋₃alkylsulfanyl” refers to a radical of the formula —SRₐ wherein Rₐ is a C₁₋₃alkyl radical as generally defined above.

As used herein, the term “C₁₋₃alkylsulfonyl” refers to a radical of the formula —S(O)₂Rₐ wherein Rₐ is a C₁₋₃alkyl radical as generally defined above.

The presence of one or more possible asymmetric carbon atoms in a compound of Formula (I) means that the compounds may occur in chiral isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of Formula (I). Likewise, Formula (I) is intended to include all possible tautomers (including lactam-lactim tautomerism and keto-enol tautomerism) where present. The present invention includes all possible tautomeric forms for a compound of Formula (I).

In each case, the compounds of Formula (I) according to the invention are in free form, in oxidized form as an N-oxide, in covalently hydrated form, or in salt form, e.g., an agronomically usable or agrochemically acceptable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following lists provide definitions, including preferred definitions, for substituents L¹, R¹, R², R³, R⁴, R⁵, R⁶, R⁷, R⁸ and R⁹ with reference to the compounds of Formula (I) of the present invention. For any one of these substituents, any of the definitions given below may be combined with any definition of any other substituent given below or elsewhere in this document.

L¹ is —O— or —CH₂— or a bond. In certain embodiments of the invention, L¹ is —O—. In certain embodiments of the invention, L¹ is —CH₂—. In certain embodiments of the invention, L¹ is a bond.

R¹ is selected from hydrogen, halogen, cyano, C₁-C₃-alkyl or C₁-C₃-alkoxy. Preferably, R¹ is hydrogen, chloro, methyl or methoxy. Most preferably, R¹ is hydrogen. In embodiments of the invention, R¹ is selected from C₁-C₃-alkyl or C₁-C₃-alkoxy, such as methyl or methoxy.

R² is selected from hydrogen, cyano, mercaptyl, halogen, C₁-C₃-alkyl, C₂-C₃-alkenyl, C₂-C₃-alkynyl, C₁-C₃-alkoxy, C₁-C₃-fluoroalkyl, C₁-C₃-fluoroalkoxy, C₃-C₄-cycloalkyl or C₁-C₃-alkylsulfonyl. R² may also be selected from hydrogen, cyano, mercaptyl, halogen, C₁-C₃-alkyl, C₁-C₃-alkoxy, C₁-C₃-fluoroalkyl, C₁-C₃-fluoroalkoxy, C₃-C₄-cycloalkyl or C₁-C₃-alkylsulfonyl. Preferably, R² is hydrogen, cyano, mercaptyl, halogen, methyl, methoxy, trifluoromethyl, cyclopropyl or methylsulfonyl. Most preferably, R² is hydrogen, chloro or methyl. In embodiments of the invention, R² is a C₁-C₃-fluoroalkoxy.

R³ is selected from hydrogen, halogen, methyl, methoxy, or cyano. Preferably, R³ is hydrogen, fluoro, chloro, bromo, methoxy or cyano. Most preferably, R³ is hydrogen. R³ may also be hydrogen, halogen, cyano, methyl, C₁-C₄-alkyl, C₁-C₃-fluoroalkyl, C₂-C₄-alkenyl, C₂-C₄-alkynyl, C₁-C₄-alkoxy, C₂-C₄-alkenyloxy, C₂-C₅-alkynyloxy, C₁-C₃-fluoroalkoxy, C₃-C₄-cycloalkyl. The C₁-C₄-alkyl, C₁-C₃-fluoroalkyl, C₂-C₄-alkenyl, C₂-C₄-alkynyl, C₁-C₄-alkoxy, C₂-C₄-alkenyloxy, C₂-C₅-alkynyloxy, and C₁-C₃-fluoroalkoxy may be straight or branched.

R³ may also be phenoxy or phenoxy substituted with one or two substituents independently selected from halogen, mercapto, amino, cyano, methyl, ethyl, propyl, isopropyl, difluoromethyl, trifluoromethyl, methoxy, ethoxyR³ may also be phenoxy or phenoxy substituted with one substituent selected from halogen, mercapto, amino, cyano, methyl, ethyl, propyl, isopropyl, difluoromethyl, trifluoromethyl, methoxy, ethoxy.

R⁴ and R⁵ are independently selected from hydrogen, cyano or methyl. Preferably, R⁴ and R⁵ are hydrogen. Otherwise, R⁴ and R⁵ are independently selected from hydrogen, and cyano. Otherwise, R⁴ and R⁵, together with the carbon atom to which they are attached, form a cyclopropyl or cyclobutyl group.

R⁶ and R⁷ are independently selected from hydrogen, fluoro, chloro, methyl, trifluoromethyl, hydroxy, methoxy, methoxymethyl, difluoromethoxy, mercaptyl, methylsulfanyl. Preferably, R⁶ is hydrogen, fluoro, methyl or methoxy and R⁷ is hydrogen or fluoro. Most preferably, R⁶ is hydrogen or fluoro and R⁷ is hydrogen or fluoro. Otherwise, R⁶ and R⁷, together with the carbon atom to which they are attached, form an oxo, cyclopropyl, or cyclobutyl group, or a 4- to 6-membered monocyclic heterocycle group which comprises 1 or 2 heteroatoms individually selected from N, O and S, preferably they form, together with the carbon atom to which they are attached, a cyclopropyl, or cyclobutyl group.

R⁴ and R⁶, together with the carbon atom to which they are attached, may form a C₃-C₄-cycloalkyl, such as a cyclopropyl, or a 4- to 6-membered monocyclic heterocycle group which comprises 1 or 2 heteroatoms individually selected from N, O and S, and R⁵ and R⁷ are independently hydrogen or halogen.

R⁸ is phenyl optionally substituted with:
(a) a single substituent selected from C₁-C₄-alkyl, C₁-C₂-haloalkyl, C₁-C₃-alkoxy, C₁-C₃-alkyl sulfanyl, C₁-C₃-alkyl sulfonyl, C₁-C₂-haloalkoxy, C₂-C₃-alkenyl, C₂-C₃-haloalkenyl, C₂-C₃- alkynyl, C₃-C₄-cycloalkyl, C₃-C₄-cycloalkyloxy or —NH(COC₁-C₃-alkyl); or
(b) 1, 2 or 3 substituents that may be the same or different, independently selected from hydroxyl, halogen, mercapto, amino, cyano, methyl, ethyl, propyl, isopropyl, difluoromethyl, trifluoromethyl, methoxy, ethoxy, propyloxy, isopropyloxy, methylsulfanyl, methylsulfonyl, difluoromethoxy, trifluoromethoxy, cyclopropyl, cyclobutyl, cyclopropyloxy, —NH(COCH₃).

Preferably, R⁸ is phenyl optionally substituted as defined for (b) above, wherein there may be 1 or 2 substituents that may be the same or different. More preferably, R⁸ is phenyl optionally substituted by 1 or 2 substituents independently selected from hydroxyl, halogen, cyano, methyl, ethyl, difluoromethyl, trifluoromethyl, methoxy or ethoxy. Most preferably, R⁸ is phenyl optionally substituted by 1 or 2 substituents independently selected from halogen, in particular chloro, eg, R⁸ is 2,4-dichlorophenyl.

R⁹ is phenyl optionally substituted with:
a single substituent selected from C₁-C₄-alkyl, C₁-C₂-haloalkyl, C₁-C₄-alkoxy, C₁-C₃-alkylsulfanyl, C₁-C₃-alkylsulfonyl, C₁-C₂-haloalkoxy, C₂-C₃-alkenyl, C₂-C₃-haloalkenyl, C₂-C₃-alkynyl, C₃-C₄-cycloalkyl, C₃-C₄-cycloalkyloxy; or
1, 2 or 3 substituents which may be the same or different, independently selected from hydroxyl, halogen, mercapto, amino, cyano, methyl, ethyl, propyl, isopropyl, difluoromethyl, trifluoromethyl, methoxy, ethoxy, propyloxy, iso-propyloxy, methylsulfanyl, methylsulfonyl, difluoromethoxy, trifluoromethoxy, cyclopropyl, cyclobutyl, cyclopropyloxy, —NH(COCH₃).

Preferably, R⁹ is phenyl optionally substituted with 1 or 2 substituents independently selected from hydroxyl, halogen, mercapto, amino, cyano, methyl, ethyl, propyl, isopropyl, difluoromethyl, trifluoromethyl, methoxy, ethoxy, propyloxy, iso-propyloxy, t-butoxy, propynoxy, methylsulfanyl, methylsulfonyl, difluoromethoxy, trifluoromethoxy, cyclopropyl, cyclobutyl, cyclopropyloxy and —NH(COCH₃).

More preferably, R⁹ is phenyl, optionally substituted with 1 or 2 substituents independently selected from halogen, cyano, methyl, ethyl, difluoromethyl, trifluoromethyl, methoxy, ethoxy, propyloxy, iso-propyloxy, t-butoxy, propynoxy, methylsulfanyl, difluoromethoxy, trifluoromethoxy, cyclopropyl and —NH(COCH₃). Even more preferably, R⁹ is phenyl optionally substituted by methyl, trifluoromethyl or cyclopropyl, in particular, in the 3-position. Most preferably, R⁹ is phenyl substituted by methyl or cyclopropyl, eg, 3-methylphenyl or 3-cyclopropylphenyl.

Preferably, the compound according to Formula (I) is selected from a compound 1.1 to 1.98 listed in Table T1 (below); for instance a compound 1.1 to 1.88 listed in Table T1 (below); such as a compound selected from compounds 1.1, 1.2, 1.4, 1.5, 1.6, 1.7, 1.8, 1.9, 1.10, 1.11, 1.12, 1.13, 1.14, 1.15, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.43, 1.44, 1.45, 1.46, 1.47, 1.48, 1.49, 1.50, 1.51, 1.52, 1.53, 1.54, 1.55, 1.56, 1.57, 1.58, 1.59, 1.62, 1.63, 1.64, 1.65, 1.66, 1.67, 1.68, 1.69, 1.71, 1.72, 1.73, 1.74, 1.75, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, listed in Table T1 (below). For instance, the compound according to Formula (I) is compound 1.1 or compound 1.22 listed in Table T1 (below).

The compounds of the present invention may be enantiomers of the compound of Formula (I) as represented by Formula (I-1) or Formula (I-2) or Formula (I-3) or Formula (I-4), wherein R⁴ and R⁵ are different substituents and/or wherein R⁶ and R⁷ are different substituents.

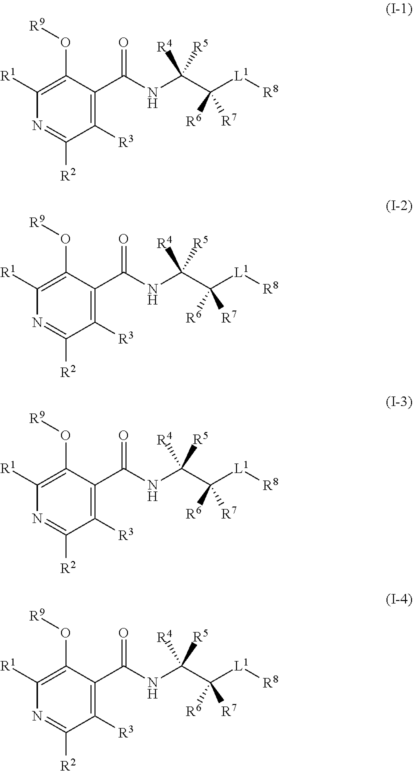

The compounds of formula (I) according to the invention can be made as shown in the following schemes 1 to 13, wherein L¹, R¹, R², R³, R⁴, R⁵, R⁶, R⁷, R⁸, and R⁹ are as defined for a compound of formula (I), unless otherwise stated.

The compounds of formula (I) are obtained by an amide-coupling transformation with compounds of formula (II), wherein X is OH, and amine compounds of formula (III) by activating the carboxylic acid function of the compounds of formula (II), a process that usually takes place by converting the —OH of the carboxylic acid into a good leaving group, such as a chloride group, for example by using (COCl)₂ or SOCl₂, prior to treatment with the compounds of formula (III), preferably in a suitable solvent (e.g., N-methylpyrrolidone, acetonitrile, dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile) optionally in the presence of a base (e.g., triethylamine or N,N-diisopropylethylamine). For examples, see Chem. Soc. Rev. (2009), 38, 606 and Chem. Soc. Rev. (2011), 40, 5084. Compounds of formula (II) and compounds of formula (III) are either known or commercially available. This is shown in Scheme 1.

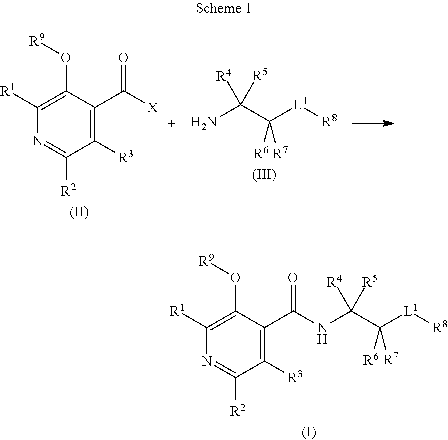

Compounds of formula (I), are prepared from reacting nucleophilic compounds of formula (IV) with electrophilic compounds of formula (V), wherein Y is a suitable leaving group such as fluoro, chloro, bromo, iodo, BF₃K, B(OH)₂ or B(pinacol), in the presence of base (e.g. KO-t-Bu, K₃PO₄, K₂CO₃, triethylamine, or Cs₂CO₃), in a suitable solvent (e.g. N-methylpyrrolidone, dimethylacetamide, acetonitrile, tetrahydrofuran, 2-methyl tetrahydrofuran, sulfolane, or dimethylsulfoxide) at temperatures between 60° C. and 110° C. and preferably using a metal catalyst complex (e.g. Cu or Pd). For related examples, see Eur. J. Org. Chem., (2011), 18, 3353; J. Org. Chem., (2009), 74, 7951; Tetrahedron Lett., (2012), 53, 5318. Compounds of formula (IV) are either known or commercially available. This is shown in Scheme 2.

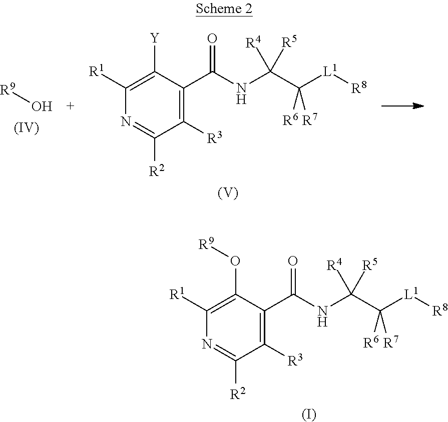

Compounds of formula (II), wherein X is C₁-C₄-alkoxy, are prepared from reacting nucleophilic compounds of formula (IV) with electrophilic compounds of formula (VI), wherein X is OH or C₁-C₄-alkoxy and Y is a suitable leaving group such as fluoro, chloro, bromo, iodo, BF₃K, B(OH)₂ or B(pinacol), in the presence of base (e.g. KO-t-Bu, K₃PO₄, K₂CO₃, triethylamine, or Cs₂CO₃, in a suitable solvent (e.g. N-methylpyrrolidone, dimethylacetamide, acetonitrile, tetrahydrofuran, 2-methyl tetrahydrofuran, sulfolane, dimethylsulfoxide) at temperatures between 60° C. and 110° C. and preferably using a metal catalyst complex (e.g. Cu or Pd). For related examples, see Eur. J. Org. Chem., (2011), 18, 3353; J. Org. Chem., (2009), 74, 7951; Tetrahedron Lett., (2012), 53, 5318; WO 2008/110313 and WO 2012/136604. Additionally, compounds of formula (II), wherein X=C₁-C₄-alkoxy, are readily hydrolyzed under conditions described in the literature to afford compounds of formula (II), wherein X is OH. Compounds of formula (VI) are either known or commercially available. This is shown in Scheme 3.

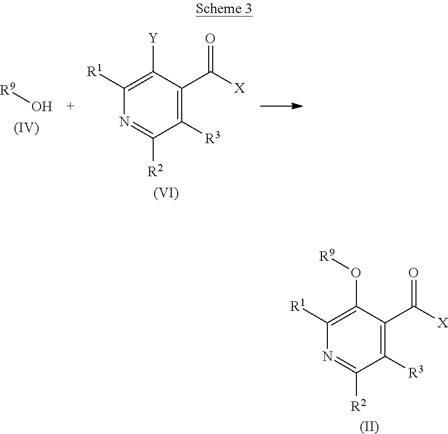

The compounds of formula (V), wherein Y is halogen or OH, can be obtained by an amide coupling transformation with compounds of formula (VI), wherein X is OH and Y is halogen or OH, and amine compounds of formula (III) by activating the carboxylic acid function of the compounds of formula (VI), a process that usually takes place by converting the —OH of the carboxylic acid into a good leaving group, such as a chloride group, for example by using (COCl)₂ or SOCl₂, prior to treatment with the compounds of formula (III), preferably in a suitable solvent (e.g., N-methylpyrrolidone dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile), optionally in the presence of a base (e.g., triethylamine or N,N-diisopropylethylamine). For examples, see Chem. Soc. Rev. (2009), 38, 606 and. Chem. Soc. Rev. (2011), 40, 5084. This is shown in Scheme 4.

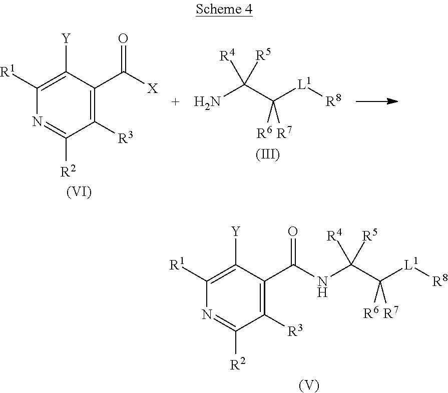

Alternatively, compounds of formula (I) are prepared by treatment of compounds of formula (VII), wherein Z is chloro, bromo, iodo with a suitable organometallic (e.g. MeMgBr, MeZnCl, AlMe₃) or organometalloid (e.g. trimethylboroxine) reagent in the presence of a suitable metal (e.g. CuBr) or catalyst complex (e.g. 1,1′-bis(diphenylphosphino)ferrocene-palladium(II)dichloride), optionally in the presence of a base (e.g. K₂CO₃) in an inert solvent such as toluene or 2-methyl tetrahydrofuran at temperatures from 80° C. to 110° C. For examples, see J. Org. Chem. (1987), 52, 3847; WO 2006/045514; WO 2004/080998. This is shown in Scheme 5 below.

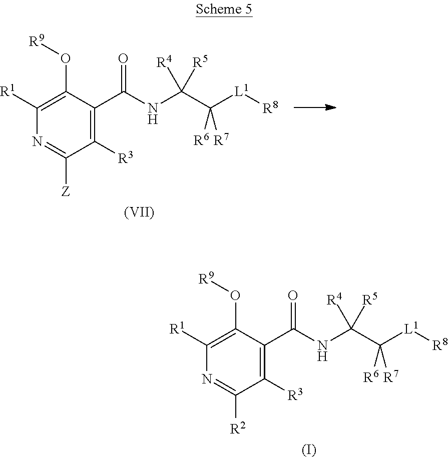

The compounds of formula (VII), wherein Z is chloro, bromo, or iodo, can be obtained by an amide-coupling transformation with compounds of formula (VIII), wherein X is OH and Z is chloro, bromo, or iodo, and amine compounds of formula (III) by activating the carboxylic acid function of the compounds of Formula (VIII), a process that usually takes place by converting the —OH of the carboxylic acid into a good leaving group, such as a chloride group, for example by using (COCl)₂ or SOCl₂, prior to treatment with the compounds of formula (III), preferably in a suitable solvent (e.g., acetonitrile, dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., MeCN), optionally in the presence of a base (e.g., triethylamine or N,N-diisopropylethylamine). For examples, see Chem. Soc. Rev. (2009), 38, 606 and Chem. Soc. Rev. (2011), 40, 5084. This is shown in Scheme 6.

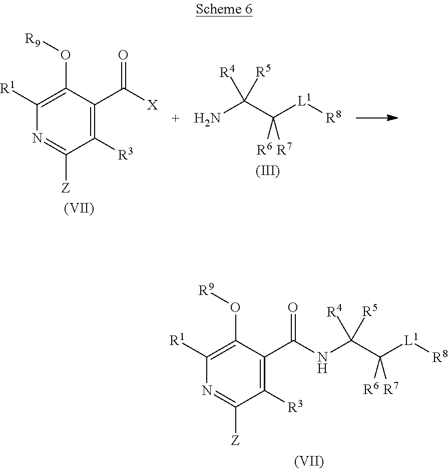

Compounds of formula (II), wherein X is OH or C₁-C₄-alkoxy are prepared by treatment of compounds of formula (VIII), wherein X is OH or C₁-C₄-alkoxy and Z is chloro, bromo, iodo with a suitable organometallic (e.g. MeMgBr, MeZnCl, AlMe₃) or organometalloid (e.g. trimethylboroxine) reagent in the presence of a suitable metal source (e.g. CuBr) or catalyst complex (e.g. 1,1′-bis(diphenylphosphino)ferrocene-palladium(II) dichloride), optionally in the presence of a base (e.g. K₂CO₃) in an inert solvent such as toluene or Me-THF at temperatures from 80° C. to 110° C. For related examples, see: J. Org. Chem. (1987), 52, 3847; WO 2006/045514; WO 2004/080998. This is shown in Scheme 7.

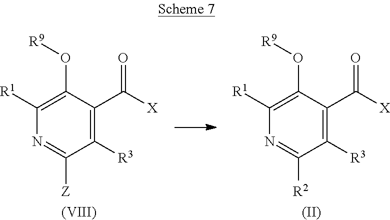

Compounds of formula (VI), wherein wherein X is OH or C₁-C₄-alkoxy and Y is OH or halogen, are prepared by treatment of compounds of formula (IX), wherein Z is bromo, iodo and Y is fluoro, chloro or bromo, preferably chloro, with a suitable organometallic (e.g. MeMgBr, MeZnCl, AlMe₃) or organometalloid (e.g. trimethylboroxine) reagent in the presence of a suitable metal source (e.g. CuBr) or catalyst complex (e.g. 1,1′-bis(diphenylphosphino)ferrocene-palladium(II) dichloride), optionally in the presence of a base (e.g. K₂CO₃) in an inert solvent such as toluene or 2-methyl tetrahydrofuran at temperatures from 60° C. to 110° C. J. Org. Chem. (1987), 52, 3847; WO 2006/045514; WO 2004/080998. Compounds of formula (IX) are either known or commercially available. This is shown in Scheme 8.

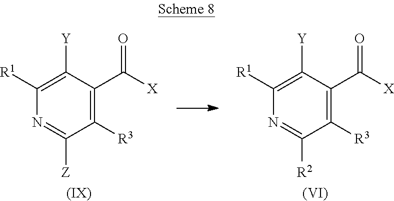

Compounds of formula (VIII), wherein X is OH or C₁-C₄-alkoxy and Z is chloro or bromo, are prepared from reacting nucleophilic compounds of formula (IV) with electrophilic compounds of formula (IX), wherein X is OH or C₁-C₄-alkoxy and Y is a suitable leaving group such as fluoro, chloro, bromo, iodo, BF₃K, B(OH)₂ or B(pinacol) and Z is chloro or bromo, preferably bromo, in the presence of base (e.g. KO-t-Bu, K₂CO₃, triethylamine, or Cs₂CO₃), in a suitable solvent (e.g. acetonitrile, tetrahydrofuran, 2-methyltetrahydrofuran, N-methylpyrrolidone, dimethylacetamide) at temperatures between 60° C. and 110° C. and preferably using a metal catalyst complex (e.g. Cu and Pd). For related examples, see Eur. J. Org. Chem., (2011), 18, 3353; J. Org. Chem., (2009), 74, 7951; Tetrahedron Lett., (2012), 53, 5318. Compounds of formula (IX) are either known or commercially available. This is shown in Scheme 9.

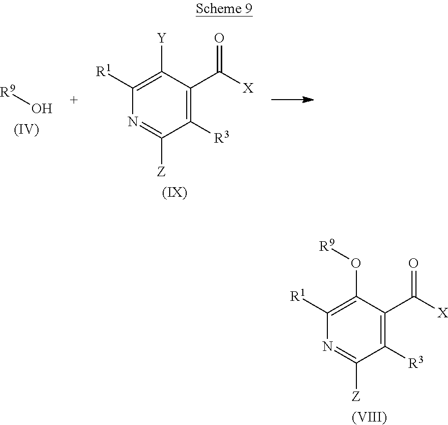

Compounds of formula (II), wherein X is OH or C₁-C₄-alkoxy, are prepared from reacting nucleophilic compounds of formula (VI), wherein Y is OH and X is OH or C₁-C₄-alkoxy, with electrophilic compounds of formula (X), wherein and E is fluoro, chloro, bromo, iodo, BF₃K, B(OH)₂ or B(pinacol), in a suitable solvent (e.g. dichloromethane, 1,2-dichloromethane, acetonitrile, tetrahydrofuran, 2-methyl tetrahydrofuran, N-methylpyrrolidone, dimethylacetamide) at temperatures between 40° C. and 80° C. and using a metal source (e.g. Cu(OAc)₂), and preferably in the presence of an oxidant such as O₂ or a suitable palladium pre-catalyst, such as RockPhos Pd G3, in the presence of a base (e.g. K₃PO₄) and suitable solvent (e.g. dimethyl ether or toluene) at temperatures between 20° C. and 80° C. For related examples, see Org. Lett., (2003), 5, 1381; Tetrahedron Lett. (1998), 39, 2933; Tetrahedron Lett., (2003), 44, 3863; and Org. Lett., (2013), 15, 2876. Compounds of formula (X) and compounds of formula (VI) are either known or commercially available. This is shown in Scheme 10.

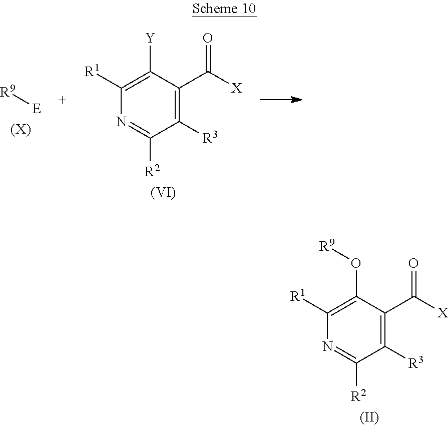

Compounds of formula (I) are prepared from reacting nucleophilic compounds of formula (V), wherein Y is OH, with electrophilic compounds of formula (X), wherein and E is chloro, bromo, iodo, BF₃K, B(OH)₂ or B(pinacol), in a suitable solvent (e.g. dichloromethane, 1,2-dichloromethane, acetonitrile, tetrahydrofuran, 2-methyl tetrahydrofuran) at temperatures between 40° C. and 80° C. and using a metal source (e.g. Cu(OAc)₂), optionally in the presence of an oxidant such as O₂ or a suitable palladium pre-catalyst, such as RockPhos Pd G3, in the presence of a base (e.g. K₃PO₄) and suitable solvent (e.g. dimethyl ether or toluene) at temperatures between 20° C. and 80° C. For related examples, see Org. Lett., (2003), 5, 1381; Tetrahedron Lett. (1998), 39, 2933; Tetrahedron Lett., (2003), 44, 3863; and Org. Lett., (2013), 15, 2876. Compounds of formula (X) are either known or commercially available. This is shown in Scheme 11.

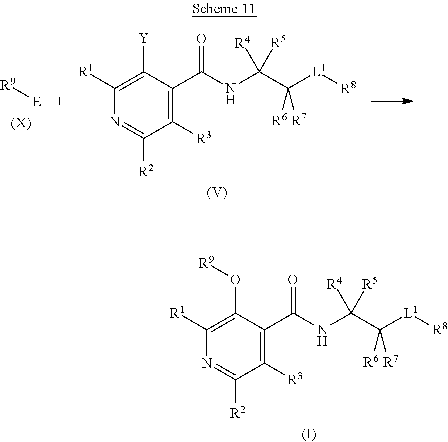

Compounds of formula (II), wherein X is C₁-C₄-alkoxy and R³ is C₁-C₄-alkoxy, C₂-C₄-alkenyloxy, C₂-C₄-alkynyloxy, C₁-C₃-fluoroalkoxy, C₃-C₄-cycloalkoxycycloalkyl or phenoxy are prepared by treatment of compounds of formula (XI), Z is halogen with a compound of formula (XII) R^{z}—OH, wherein R^{z} is C₁-C₄-alkoxy, C₂-C₄-alkenyloxy, C₂-C₄-alkynyloxy, C₁-C₃-fluoroalkoxy, C₃-C₄-cycloalkyl or phenoxy, in the presence of a base, such as NaH, KO-t-Bu, K₃PO₄, K₂CO₃, triethylamine, or Cs₂CO₃, in a suitable solvent (e.g. N-methylpyrrolidone, dimethylacetamide, acetonitrile, tetrahydrofuran, 2-methyl tetrahydrofuran, sulfolane, dimethylsulfoxide) at temperatures between 20-120° C. to give compounds of formula (II). For related examples, see: F. Terrier, Modern Nucleophilic Aromatic Substitution, Wiley-VCH, Weinheim, 2013. Additionally, compounds of formula (II), wherein X=C₁-C₄-alkoxy, are readily hydrolyzed under conditions described in the literature to afford compounds of formula (II), wherein X is OH. This is shown in Scheme 12

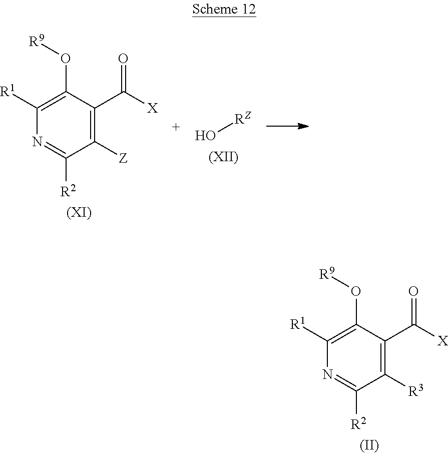

Alternatively, compounds of formula (II), wherein R³ is C₁-C₄-alkoxy, C₂-C₄-alkenyloxy, C₂-C₄-alkynyloxy, C₁-C₃-fluoroalkoxy, C₃-C₄-cycloalkoxylcycloalkyl, are prepared by treatment of compounds of formula (XI), wherein Z is OH, with a compound of formula (XIII) R^{z}-LG, wherein R^{z} is as defined above in connection with Scheme 12, LG is a suitable leaving group, such as bromo or chloro, in the presence of a base, such as NaH, KO-t-Bu, K₃PO₄, K₂CO₃, triethylamine, or Cs₂CO₃ in a suitable solvent e.g. N-methylpyrrolidone, dimethylacetamide, acetonitrile, tetrahydrofuran, 2-methyl tetrahydrofuran, sulfolane, dimethylsulfoxide) at temperatures between 20° C. and 100° C. This is shown in Scheme 13

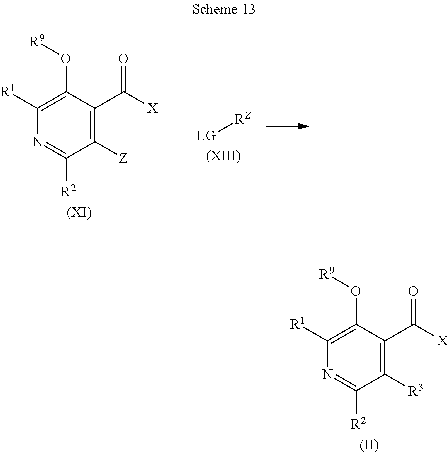

As already indicated, surprisingly, it has now been found that the compounds of Formula (I) of the present invention have, for practical purposes, a very advantageous level of biological activity for protecting plants against diseases that are caused by fungi.

The compounds of Formula (I) can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of Formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of Formula (I) is applied to the plants, to parts thereof or the locus thereof.

It is also possible to use compounds of Formula (I) as a fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of Formula (I) as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of Formula (I) before planting: seed, for example, can be dressed before being sown. The active compounds of Formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of Formula (I) can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of Formula (I) are for example, effective against fungi and fungal vectors of disease as well as phytopathogenic bacteria and viruses. These fungi and fungal vectors of disease as well as phytopathogenic bacteria and viruses are for example:
Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. inclusing B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp,. Stereum hirsutum, Thanatephorus cucumeris, Thielaviopsis basicola, Tilletia spp, Trichoderma spp. including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp.

The compounds of Formula (I) may be used for example on turf, ornamentals, such as flowers, shrubs, broad-leaved trees or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO93/07278, WO95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup@ (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of Formula (I) may be used in unmodified form or, preferably, together with the adjuvants conventionally employed in the art of formulation. To this end they may be conveniently Formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular Formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of Formula (I) are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of Formula (I) may be used in the form of (fungicidal) compositions for controlling or protecting against phytopathogenic microorganisms, comprising as active ingredient at least one compound of Formula (I) or of at least one preferred individual compound as defined herein, in free form or in agrochemically usable salt form, and at least one of the above-mentioned adjuvants.

The invention therefore provides a composition, preferably a fungicidal composition, comprising at least one compound Formula (I) an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally-active compounds, for example an additional fungicidal active ingredient in addition to the compound of Formula (I).

The compound of Formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Specific examples of suitable additional active ingredients also include a compound selected from the group of substances consisting of petroleum oils, 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxafos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chino-methionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fen-pyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1 -yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B₁, trimedlure B₂, trimedlure C, trunc-call, 2-(octylthio)ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, El 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla), schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, and meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxo1-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibencon-azole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimetho-morph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad, fenhexamid, fosetyl-aluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1,3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, tebufloquin, ipflufenoquin, quinofumelin, isofetamid, N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1,1,3-trimethylindan-4- yl) pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N-((3R)-1,1,3-trimethylindan-4-yl)pyridine-3- arboxamide,α-(1,1-dimethylethyl)-α-[4′-(trifluoromethoxy)[1,1′-biphenyl]-4-yl]-5-pyrimidinemethanol, fluoxapiprolin, enoxastrobin, 4-[[6-[2-(2,4-difluorophenyl)-1,1-difluoro-2-hydroxy-3-(1,2,4-triazol-1-yl)propyl]-3-pyridyl]oxy] benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile, trinexapac, coumoxystrobin, zhongshengmycin, thiodiazole copper, zinc thiazole, amectotractin, iprodione, seboctylamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl)propan-2-ol (this compound may be prepared from the methods described in WO 2017/029179); 2-[6-(4-bromophenoxy)-2-(trifluoromethyl)-3-pyridyl]-1-(1,2,4-triazol-1-yl)propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281) N-methyl-4-[5-(trifluoromethyl)-1,2,4-oxadiazol-3-yl]benzenecarboth ioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N-[N-methoxy-C-methyl-carbonimidoyl]-4-[5-(trifluoromethyl)-1,2,4-oxadiazol-3-yl]benzamide (these compounds may be prepared from the methods described in WO 2018/202428), fluoxapiprolin, enoxastrobin, trinexapac, coumoxystrobin.

N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine. The compounds in this paragraph may be prepared from the methods described in WO 2017/055473, WO 2017/055469, WO 2017/093348 and WO 2017/118689. 2-[6-(4-chlorophenoxy)-2-(trifluoromethyl)-3-pyridyl]-1-(1,2,4-triazol-1-yl)propan-2-ol (this compound may be prepared from the methods described in WO 2017/029179), 2-[6-(4-bromophenoxy)-2-(trifluoromethyl)-3-pyridyl]-1-(1,2,4-triazol-1-yl)propan-2-ol (this compound may be prepared from the methods described in WO 2017/029179), 3-[2-(1-chlorocyclopropyl)-3-(2-fluorophenyl)-2-hydroxy-propyl]imidazole-4-carbonitrile (this compound may be prepared from the methods described in WO 2016/156290), 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290).
(4-phenoxyphenyl)methyl 2-amino-6-methyl-pyridine-3-carboxylate (this compound may be prepared from the methods described in WO 2014/006945), 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281), N-methyl-4-[5-(trifluoromethyl)-1,2,4-oxadiazol-3-yl]benzenecarbothioamide, N-methyl-4-[5-(trifluoromethyl)-1,2,4-oxadiazol-3-yl]benzamide, (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707), N′-(2-chloro-5-methyl-4-phenoxy-phenyl)-N-ethyl-N-methyl-formamidine, N′-[2-chloro-4-(2-fluorophenoxy)-5-methyl-phenyl]-N-ethyl-N-methyl-formamidine (this compound may be prepared from the methods described in WO 2016/202742), 2-(difluoromethyl)-N-[(3S)-3-ethyl-1,1-dimethyl-indan-4-yl]pyridine-3-carboxamide (this compound may be prepared from the methods described in WO 2014/095675), zhongshengmycin, thiodiazole copper, zinc thiazole, amectotractin, iprodione, cyclobutrifluram, flubeneteram, anisiflupurin.
N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1 R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO 2017/153380).
1-(6,7-dimethylpyrazolo[1,5-a]pyridin-3-yl)-4,4,5-trifluoro-3,3-dimethyl-isoquinoline, dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H cyclopenta[e]benzimidazole (these compounds may be prepared from the methods described in WO 2016/156085.
N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifuoromethyptetrahydrofuran-2-yl]phenyl]-N-methyl-formamidine (these compounds may be prepared from the methods described in WO2019/110427).
(5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414);
ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N-[N-methoxy-C-methyl-carbonimidoyl]-4-[5-(trifluoromethyl)-1,2,4-oxadiazol-3-yl]benzamide (these compounds may be prepared from the methods described in WO 2018/202428); N-(2-fluorophenyl)-4-[5-(trifluoromethyl)-1,2,4-oxadiazol-3-yl]benzamide, [(1S,2S)-1-methyl-2-(o-tolyl)propyl] (2S)-2-[(4-methoxy-3-propanoyloxy-pyridine-2-carbonyl)amino]propanoate, seboctylamine, chloroinconazide, flumetylsulforim, fluoxytioconazole.

The following mixtures of the compounds of Formula (I) with active ingredients are preferred. The abbreviation “TX” means one compound selected from the group consisting of the compounds as represented in Tables A-1 to A-48 or B-1 to B-40 (below), or a compound 1.1 to 1.98 listed in Table T1 (below):
a compound selected from the group of substances consisting of petroleum oils+TX, 1,1-bis(4-chlorophenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfo +TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX , Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalur +TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B₂+TX, trimedlure C+TX, trunc-call+TX, 2-(octylthio)ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla)+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, and meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, 2-fluoro-N-(3-methoxyphenyl)-9H-purin-6-amine+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX ,acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxo1-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl-+TX, Rmetalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorothalonil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, fluopicolide+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1,3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenox]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, tebufloquin+TX, ipflufenoquin+TX, quinofumelin+TX, isofetamid+TX, N-[2-[2,4-dichloro-phenox]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2- (difluoromethyl) -N-((3R)-1,1,3-trimethylindan-4-yl)pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl) -N-((3R)-1,1,3-trimethylindan-4-yl)pyridine-3-carboxamide+TX, α-(1,1-dimethylethyl) -α- [4′- (trifluoromethoxy)[1,1′-biphenyl]- 4- yl]-5- pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, N-octyl-N′-[2-(octylamino)ethyl]ethane-1,2-diamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[2-trifluoromethyl)oxetan-2-yl]phenyl]-N-methyl-formamidine+TX, N-ethyl-N′-[5-methoxy-2-methyl-4-[2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1 R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl) -8-fluoro-quinoline-3-carboxamide+TX (these compounds may be prepared from the methods described in WO2017/153380);
   1-(6,7-dimethylpyrazolo[1,5-a]pyridin-3-yl)-4,4,5-trifluoro-3,3-dimethyl-isoguinoline+TX, 1-(6,7-dimethylpyrazolo[1,5-a]pyridin-3-yl)-4,4,6-trifluoro-3,3-dimethyl-isoguinoline+TX, 4,4-difluoro-3,3-dimethyl-1-(6-methylpyrazolo[1,5-a]pyridin-3-yl)isoguinoline+TX, 4,4-difluoro-3,3-dimethyl-1-(7-methylpyrazolo[1,5-a]pyridin-3-yl)isoguinoline+TX, 1-(6-chloro-7-methyl-pyrazolo[1,5-a]pyridin-3-yl)-4,4-difluoro-3,3-dimethyl-isoguinoline+TX (these compounds may be prepared from the methods described in WO2017/025510); 1-(4,5-dimethylbenzimidazol-1-yl)-4,4,5-trifluoro-3,3-dimethyl-isoguinoline+TX, 1-(4,5-dimethylbenzimidazol-1-yl)-4,4-difluoro-3,3-dimethyl-isoguinoline+TX, 6-chloro-4,4-difluoro-3,3-dimethyl-1-(4-methylbenzimidazol-1-yl)isoguinoline+TX, 4,4-difluoro-1-(5-fluoro-4-methyl-benzimidazol-1-yl)-3,3-dimethyl-isoguinoline+TX, 3-(4,4-difluoro-3,3-dimethyl-1-isoguinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[N-methoxy-C-methyl-carbonimidoyl]-4-[5-(trifluoromethyl)-1,2,4-oxadiazol-3-yl]benzamide+TX (these compounds may be prepared from the methods described in WO 2018/202428), chloroinconazide+TX, seboctylamine+TX, chloroinconazide+TX, flumetylsulforim+TX, fluoxytioconazole+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright© 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula (I) selected from one compound as represented in Tables A-1 to A-48 or B-1 to B-40 (below) or a compound 1.1 to 1.98 listed in Table T1 (below) is preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound as represented in Tables A-1 to A-48 or B-1 to B-40 (below), or a compound 1.1 to 1.98 listed in Table T1 (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying a compound as represented in Tables A-1 to A-48 or B-1 to B-40 (below), or a compound 1.1 to 1.98 listed in Table T1 (below) and the active ingredient(s) as described above, is not essential for working the present invention.

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP- 357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO 96/15121 and also with anthelmintic active cyclic depsipeptides such as those described in WO 96/11945, WO 93/19053, WO 93/25543, EP 0 626 375, EP 0 382 173, WO 94/19334, EP 0 382 173, and EP 0 503 538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO 95/19363 or WO 04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos- methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1 R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195,RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of the invention is related to the use of a compound of Formula (I) or of a preferred individual compound as defined herein, of a composition comprising at least one compound of Formula (I) or at least one preferred individual compound as above-defined, or of a fungicidal or insecticidal mixture comprising at least one compound of Formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of the invention is related to a method of controlling or preventing an infestation of plants, e.g., useful plants such as crop plants, propagation material thereof, e.g. seeds, harvested crops, e.g., harvested food crops, or of non-living materials by insects or by phytopathogenic or spoilage microorganisms or organisms potentially harmful to man, especially fungal organisms, which comprises the application of a compound of Formula (I) or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of Formula (I), or an agrochemical composition which contains at least one of said compounds, is foliar application. The frequency of application and the rate of application will depend on the risk of infestation by the corresponding pathogen or insect. However, the compounds of Formula (I) can also penetrate the plant through the roots via the soil (systemic action) by drenching the locus of the plant with a liquid Formulation, or by applying the compounds in solid form to the soil, e.g. in granular form (soil application). In crops of water rice such granulates can be applied to the flooded rice field. The compounds of Formula (I) may also be applied to seeds (coating) by impregnating the seeds or tubers either with a liquid formulation of the fungicide or coating them with a solid formulation.

A formulation, e.g. a composition containing the compound of Formula (I), and, if desired, a solid or liquid adjuvant or monomers for encapsulating the compound of Formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of Formula (I) per kg of seed, preferably from 0.01 to 10 g per kg of seed are generally sufficient.

Suitably, a composition comprising a compound of Formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingre-dients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly Formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of Formula (I) optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

The compounds according to the following Tables A-1 to A-48 and B-1 to B-40 below can be prepared according to the methods described above. The examples which follow are intended to illustrate the invention and show preferred compounds of formula (I).

Table A: This table discloses 64 substituent definitions (G) of compounds of the formula (Ia) according to the invention:

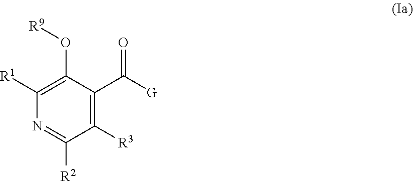

wherein G (comprising —N(H)—, R⁴, R⁵, R⁶, R⁷, R⁸ and L¹) according to compounds of Formula (I) of the invention is

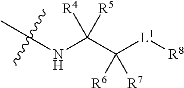

and wherein the G substituents are as defined below:

| | G |
|---|---|
| 1 | [2-fluoro-2-[3- |
| | (trifluoromethyl)phenyl]ethyl]amino |
| 2 | [2-(3-chlorophenyl)-2-fluoro-propyl]amino |
| 3 | [2-(2,4-dichlorophenyl)-1-methyl-2- |
| | methylsulfanyl-ethyl]amino |
| 4 | [2-(2,6-dichlorophenyl)-2-hydroxy- |
| | ethyl]amino |
| 5 | [2-(2,4-difluorophenyl)-2,2-difluoro- |
| | ethyl]amino |
| 6 | [2-(4-chlorophenyl)-2-hydroxy-1-methyl- |
| | ethyl]amino |
| 7 | [(1R,2S)-2-hydroxy-1-methyl-2-phenyl- |
| | ethyl]amino |
| 8 | [2-(4-chlorophenyl)-2-hydroxy- |
| | ethyl]amino |
| 9 | [(1S,2R)-2-hydroxy-1-methyl-2-phenyl- |
| | ethyl]amino |
| 10 | (2-hydroxy-1-methyl-2-phenyl- |
| | ethyl)amino |
| 11 | (2,2-difluoro-2-phenyl-ethyl)amino |
| 12 | (2-hydroxy-2-phenyl-ethyl)amino |
| 13 | [2-hydroxy-2-[4- |
| | (trifluoromethyl)phenyl]ethyl]amino |
| 14 | [2-(2,4-dichlorophenyl)-2-fluoro- |
| | propyl]amino |
| 15 | [1-(4- |
| | fluorophenyl)cyclobutyl]methylamino |
| 16 | [2-(2-chlorophenyl)-2-methyl- |
| | propyl]amino |
| 17 | 2-(2-chlorophenyl)ethylamino |
| 18 | 2-(4-methoxyphenyl)ethylamino |
| 19 | 2-(2-cyanophenyl)ethylamino |
| 20 | 2-(4-fluorophenyl)ethylamino |
| 21 | [(1R,2R)-2-(4- |
| | fluorophenyl)cyclopropyl]amino |
| 22 | 2-[3-(trifluoromethyl)phenyl]ethylamino |
| 23 | [2-(2,4-dichlorophenyl)-2-fluoro- |
| | ethyl]amino |
| 24 | [1-(3,4- |
| | dichlorophenyl)cyclopropyl]methylamino |
| 25 | 2-(4-cyanophenyl)ethylamino |
| 26 | [2-(2,4-dichlorophenyl)-2-methoxy-1- |
| | methyl-ethyl]amino |
| 27 | [2-(2-chlorophenyl)-2-methoxy- |
| | ethyl]amino |
| 28 | [2-(3-chlorophenyl)-2-methyl- |
| | propyl]amino |
| 29 | 2-(4-chlorophenyl)ethylamino |
| 30 | [1-[(4- |
| | chlorophenyl)methyl]cyclopropyl]amino |
| 31 | (2-phenyl-1,3-dioxolan-2-yl)methylamino |
| 32 | 2-(p-tolyl)ethylamino |
| 33 | 2-(o-tolyl)ethylamino |
| 34 | 2-(3,5-dimethylphenyl)ethylamino |
| 35 | 2-(2,6-dimethylphenyl)ethylamino |
| 36 | 2-(3-chlorophenyl)ethylamino |
| 37 | 2-(2,6-dichlorophenyl)ethylamino |
| 38 | 2-(3,4-dichlorophenyl)ethylamino |
| 39 | 2-(2,3-dichlorophenyl)ethylamino |
| 40 | 2-(2-cyanophenyl)ethylamino |
| 41 | (1-benzylcyclopropyl)amino |
| 42 | [[1-cyano-2-(4-ethoxyphenyl)-1-methyl- |
| | ethyl]amino] |
| 43 | [1-cyano-3-(4-methoxyphenyl)-1-methyl- |
| | propyl]amino |
| 44 | (4,4,4-trifluoro-3-phenyl-butyl)amino |
| 45 | [3-(4-fluorophenyl)-1-methyl-3-oxo- |
| | propyl]amino |
| 46 | 3-(2,4-dichlorophenyl)propylamino |
| 47 | [1,1-dimethyl-3-[3- |
| | (trifluoromethyl)phenyl]propyl]amino |
| 48 | 3-(4-chlorophenyl)propylamino |
| 49 | (3-phenylpropylamino) |
| 50 | (1-methyl-3-phenyl-propyl)amino |
| 51 | (2-phenylcyclopropyl)methylamino |
| 52 | (2-phenylcyclopropyl)amino |
| 53 | (2-benzylcyclopropyl)amino |
| 54 | [3-(4-chlorophenyl)cyclobutyl]amino |
| 55 | (3-phenylcyclobutyl)amino |
| 56 | (2-phenylethylamino) |
| 57 | 2-(3,4-dimethoxyphenyl)ethylamino |
| 58 | 2-(2,4-dichlorophenyl)ethylamino |
| 59 | [2-(2,4-dichlorophenyl)-2,2-difluoro-ethyl]amino |
| 60 | [2-(2,4-dimethylphenyl)-2-fluoro-ethyl]amino |
| 61 | [2-(2,4-dimethylphenyl)-2,2-difluoro- |
| | ethyl]amino] |
| 62 | [2-(2,4-dimethylphenyl)-ethyl]amino |
| 63 | [(2S)-2-(2,4-dimethylphenyl)-2-fluoro- |
| | ethyl]amino |
| 64 | [(2S)-2-(2,4-dichlorophenyl)-2-fluoro- |
| | ethyl]amino |

The following compounds are thus specifically described in Tables A-1 to A-48 with the substituents for Formula (Ia) as follows:

Table A-1: This table provides 64 compounds A-1.01 to A-1.64 of formula (Ia) wherein R⁹ is 3-cyclopropylphenyl, R¹ is hydrogen, R² is methyl, R³ is hydrogen and G substituents are as defined in

Table A above.

For example, compound A-1.23 has the following structure:

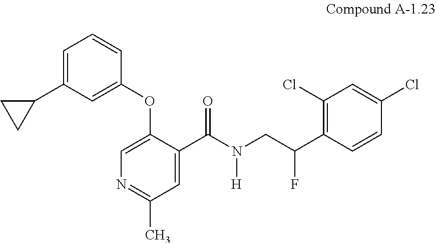 [SMILES: Cc1cc(C(=O)NCC(F)c2ccc(Cl)cc2Cl)c(Oc2cccc(C3CC3)c2)cn1]

Table A-2: This table provides 64 compounds A-2.01 to A-2.64 of formula (Ia) wherein R⁹ is 3-methylphenyl, R¹ is hydrogen, R² is methyl, R³ is hydrogen and G substituents are as defined in Table A above.
Table A-3: This table provides 64 compounds A-3.01 to A-3.64 of formula (Ia) wherein R⁹ is 3-(trifluoromethyl)phenyl, R¹ is hydrogen, R² is methyl, R³ is hydrogen and G substituents are as defined in Table A above.

For example, compound A-3.23 has the following structure:

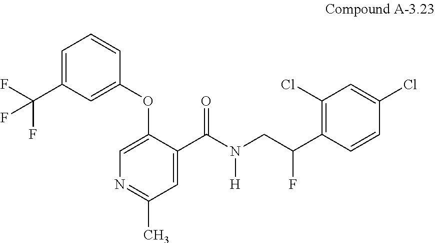 [SMILES: Cc1cc(C(=O)NCC(F)c2ccc(Cl)cc2Cl)c(Oc2cccc(C(F)(F)F)c2)cn1]

Table A-4: This table provides 64 compounds A-4.01 to A-4.64 of formula (Ia) wherein R⁹ is phenyl, R¹ is hydrogen, R² is methyl, R³ is hydrogen and G substituents are as defined in Table A above.
Table A-5: This table provides 64 compounds A-5.01 to A-5.64 of formula (Ia) wherein R⁹ is 3-cyclopropylphenyl, R¹ is hydrogen, R² is chloro, R³ is hydrogen and G substituents are as defined in Table A above.
Table A-6: This table provides 64 compounds A-6.01 to A-6.64 of formula (Ia) wherein R⁹ is 3-methylphenyl, R¹ is hydrogen, R² is chloro, R³ is hydrogen and G substituents are as defined in Table A above.
Table A-7: This table provides 64 compounds A-7.01 to A-7.64 of formula (Ia) wherein R⁹ is 3-(trifluoromethyl)phenyl, R¹ is hydrogen, R² is chloro, R³ is hydrogen and G substituents are as defined in Table A above.
Table A-8: This table provides 64 compounds A-8.01 to A-8.64 of formula (Ia) wherein R⁹ is phenyl, R¹ is hydrogen, R² is chloro, R³ is hydrogen and G substituents are as defined in Table A above.
Table A-9: This table provides 64 compounds A-9.1 to A-9.64 of formula (Ia) wherein R⁹ is 3-cyclopropylphenyl, R¹ is hydrogen, R² is hydrogen, R³ is chloro and G substituents are as defined in Table A above.

For example, compound A-9.59 has the following structure:

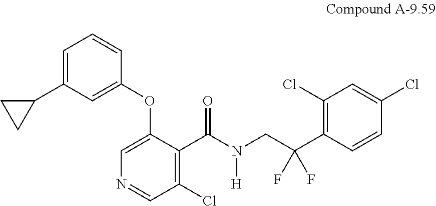 [SMILES: O=C(NCC(F)(F)c1ccc(Cl)cc1Cl)c1c(Cl)cncc1Oc1cccc(C2CC2)c1]

Table A-10: This table provides 64 compounds A-10.1 to A-10.64 of formula (Ia) wherein R⁹ is 3-methylphenyl, R¹ is hydrogen, R² is hydrogen, R³ is chloro and G substituents are as defined in Table A above.
Table A-11: This table provides 8 compounds A-11.23 and A-11.58 to A-11.64 of formula (Ia) wherein R⁹ is 3-cyclopropylphenyl, R¹ is methyl, R² is hydrogen, R³ is hydrogen and G substituents are as defined in Table A above.
Table A-12: This table provides 8 compounds A-12.23 and A-12.58 to A-12.64 of formula (Ia) wherein R⁹ is 3-methylphenyl, R¹ is methyl, R² is hydrogen, R³ is hydrogen and G substituents are as defined in Table A above.
Table A-13: This table provides 8 compounds A-13.23 and A-13.58 to A-13.64 of formula (Ia) wherein R⁹ is 3-cyclopropylphenyl, R¹ is methoxy, R² is hydrogen, R³ is hydrogen and G substituents are as defined in Table A above.
Table A-14: This table provides 8 compounds A-14.23 and A-14.58 to A-14.64 of formula (Ia) wherein R⁹ is 3-methylphenyl, R¹ is methoxy, R² is hydrogen, R³ is hydrogen and G substituents are as defined in Table A above.
Table A-15: This table provides 8 compounds A-15.23 and A-15.58 to A-15.64 of formula (Ia) wherein R⁹ is 3-cyclopropylphenyl, R¹ is hydrogen, R² is cyano, R³ is hydrogen and G substituents are as defined in Table A above.
Table A-16: This table provides 8 compounds A-16.23 and A-16.58 to A-16.64 of formula (Ia) wherein R⁹ is 3-methylphenyl, R¹ is hydrogen, R² is cyano, R³ is hydrogen and G substituents are as defined in Table A above.
Table A-17: This table provides 64 compounds A-17.1 to A-17.64 of formula (Ia) wherein R⁹ is 3-cyclopropylphenyl, R¹ is hydrogen, R² is hydrogen, R³ is hydrogen and G substituents are as defined in Table A above.
Table A-18: This table provides 64 compounds A-18.1 to A-18.64 of formula (Ia) wherein R⁹ is 3-methylphenyl, R¹ is hydrogen, R² is hydrogen, R³ is hydrogen and G substituents are as defined in Table A above.
Table A-19: This table provides 8 compounds A-19.23 and A-19.58 to A-19.64 of formula (Ia) wherein R⁹ is 3-cyclopropylphenyl, R¹ is hydrogen, R² is hydrogen, R³ is bromo and G substituents are as defined in Table A above.
Table A-20: This table provides 8 compounds A-20.23 and A-20.58 to A-20.64 of formula (Ia) wherein R⁹ is 3-methylphenyl, R¹ is hydrogen, R² is hydrogen, R³ is bromo and G substituents are as defined in Table A above.
Table A-21: This table provides 64 compounds A-21.1 to A-21.64 of formula (Ia) wherein R⁹ is 3-cyclopropylphenyl, R¹ is hydrogen, R² is hydrogen, R³ is cyano and G substituents are as defined in Table A above.
Table A-22: This table provides 64 compounds A-22.1 to A-22.64 of formula (Ia) wherein R⁹ is 3-methylphenyl, R¹ is hydrogen, R² is hydrogen, R³ is cyano and G substituents are as defined in Table A above.
Table A-23: This table provides 64 compounds A-23.1 to A-23.64 of formula (Ia) wherein R⁹ is 3-cyclopropylphenyl, R¹ is hydrogen, R² is hydrogen, R³ is fluoro and G substituents are as defined in Table A above.
Table A-24: This table provides 64 compounds A-24.1 to A-24.64 of formula (Ia) wherein R⁹ is 3-methylphenyl, R¹ is hydrogen, R² is hydrogen, R³ is fluoro and G substituents are as defined in Table A above.
Table A-25: This table provides 8 compounds A-25.23 and A-25.58 to A-25.64 of formula (Ia) wherein R⁹ is 3-cyclopropylphenyl, R¹ is hydrogen, R² is methoxy, R³ is hydrogen and G substituents are as defined in Table A above.
Table A-26: This table provides 8 compounds A-26.23 and A-26.58 to A-26.64 of formula (Ia) wherein R⁹ is 3-methylphenyl, R¹ is hydrogen, R² is methoxy, R³ is hydrogen and G substituents are as defined in Table A above.
Table A-27: This table provides 8 compounds A-27.23 and A-27.58 to A-27.64 of formula (Ia) wherein R⁹ is 3-cyclopropylphenyl, R¹ is hydrogen, R² is cyclopropyl, R³ is hydrogen and G substituents are as defined in Table A above.

For example, compound A-27.23 has the following structure:

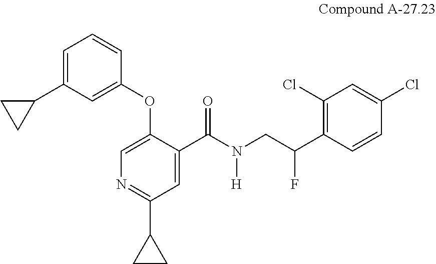 [SMILES: O=C(NCC(F)c1ccc(Cl)cc1Cl)c1cc(C2CC2)ncc1Oc1cccc(C2CC2)c1]

Table A-28: This table provides 8 compounds A-28.27 and A-28.58 to A-28.64 of formula (Ia) wherein R⁹ is 3-methylphenyl, R¹ is hydrogen, R² is cyclopropyl, R³ is hydrogen and G substituents are as defined in Table A above.
Table A-29: This table provides 8 compounds A-29.27 and A-29.58 to A-29.64 of formula (Ia) wherein R⁹ is 3-cyclopropylphenyl, R¹ is hydrogen, R² is bromo, R³ is hydrogen and G substituents are as defined in Table A above.
Table A-30: This table provides 8 compounds A-30.27 and A-30.58 to A-30.64 of formula (Ia) wherein R⁹ is 3-methylphenyl, R¹ is hydrogen, R² is bromo, R³ is hydrogen and G substituents are as defined in Table A above.
Table A-31: This table provides 8 compounds A-31.27 and A-31.58 to A-31.64 of formula (Ia) wherein R⁹ is 3-cyclopropylphenyl, R¹ is hydrogen, R² is fluoro, R³ is hydrogen and G substituents are as defined in Table A above.
Table A-32: This table provides 8 compounds A-32.27 and A-32.58 to A-32.64 of formula (Ia) wherein R⁹ is 3-methylphenyl, R¹ is hydrogen, R² is fluoro, R³ is hydrogen and G substituents are as defined in Table A above.
Table A-33: This table provides 8 compounds A-33.27 and A-33.58 to A-33.64 of formula (Ia) wherein R⁹ is 3-cyclopropylphenyl, R¹ is hydrogen, R² is trifluoromethyl, R³ is hydrogen and G substituents are as defined in Table A above.
Table A-34: This table provides 8 compounds A-34.27 and A-34.58 to A-34.64 of formula (Ia) wherein R⁹ is 3-methylphenyl, R¹ is hydrogen, R² is trifluoromethyl, R³ is hydrogen and G substituents are as defined in Table A above.
Table A-35: This table provides 8 compounds A-35.27 and A-35.58 to A-35.64 of formula (Ia) wherein R⁹ is 3-cyclopropylphenyl, R¹ is hydrogen, R² is methylsulfanyl, R³ is hydrogen and G substituents are as defined in Table A above.
Table A-36: This table provides 8 compounds A-36.27 and A-36.58 to A-36.64 of formula (Ia) wherein R⁹ is 3-methylphenyl, R¹ is hydrogen, R² is methylsulfanyl, R³ is hydrogen and G substituents are as defined in Table A above.
Table A-37: This table provides 8 compounds A-37.27 and A-37.58 to A-37.64 of formula (Ia) wherein R⁹ is 3-cyclopropylphenyl, R¹ is hydrogen, R² is methylsulfonyl, R³ is hydrogen and G substituents are as defined in Table A above.
Table A-38: This table provides 8 compounds A-38.27 and A-38.58 to A-38.64 of formula (Ia) wherein R⁹ is 3-methylphenyl, R¹ is hydrogen, R² is methylsulfonyl, R³ is hydrogen and G substituents are as defined in Table A above.
Table A-39: This table provides 8 compounds A-39.27 and A-39.58 to A-39.64 of formula (Ia) wherein R⁹ is 3-cyclopropylphenyl, R¹ is chloro, R² is methyl, R³ is hydrogen and G substituents are as defined in Table A above.
Table A-40: This table provides 8 compounds A-40.27 and A-40.58 to A-40.64 of formula (Ia) wherein R⁹ is 3-methylphenyl, R¹ is chloro, R² is methyl, R³ is hydrogen and G substituents are as defined in Table A above.
Table A-41: This table provides 8 compounds A-41.27 and A-41.58 to A-41.64 of formula (Ia) wherein R⁹ is 3-cyclopropylphenyl, R¹ is methyl, R² is chloro, R³ is hydrogen and G substituents are as defined in Table A above.
Table A-42: This table provides 8 compounds A-42.27 and A-42.58 to A-42.64 of formula (Ia) wherein R⁹ is 3-methylphenyl, R¹ is methyl, R² is chloro, R³ is hydrogen and G substituents are as defined in Table A above.
Table A-43: This table provides 64 compounds A-43.1 to A-43.64 of formula (Ia) wherein R⁹ is 3-cyclopropylphenyl, R¹ is hydrogen, R² is hydrogen, R³ is methyl and G substituents are as defined in Table A above.
Table A-44: This table provides 64 compounds A-44.1 to A-44.64 of formula (Ia) wherein R⁹ is 3-methylphenyl, R¹ is hydrogen, R² is hydrogen, R³ is methyl and G substituents are as defined in Table A above.
Table A-45: This table provides 64 compounds A-45.01 to A-45.64 of formula (Ia) wherein R⁹ is 3-cyclopropylphenyl, R¹ is hydrogen, R² is hydrogen, R³ is methoxy, and G substituents are as defined in Table A above.
Table A-46: This table provides 64 compounds A-46.01 to A-46.64 of formula (Ia) wherein R⁹ is 3-methylphenyl, R¹ is hydrogen, R² is hydrogen, R³ is methoxy, and G substituents are as defined in Table A above.
Table A-47: This table provides 64 compounds A-47.01 to A-47.64 of formula (Ia) wherein R⁹ is 3-(trifluoromethyl)phenyl, R¹ is hydrogen, R² is hydrogen, R³ is methoxy, and G substituents are as defined in Table A above.
Table A-48: This table provides 64 compounds A-48.01 to A-48.64 of formula (Ia) wherein R⁹ is phenyl, R¹ is hydrogen, R² is hydrogen, R³ is methoxy, and G substituents are as defined in Table A above.

By way of further explanation, in Tables A-1 to A-48, where a compound is identified as, eg, compound A-1.01, this defines a compound of Formula (Ia) with the R¹, R², R³ and R⁹ substituents as described in Table A-1, and the G substituent numbered “1” in Table A above, and so on for compounds A-1.02, A-1.03, etc. The same considerations apply to the compounds disclosed in Tables B-1 to B-40 below.

Table B: This table discloses 41 substituent definitions (R⁹) of compounds of the formula (Ia) according to the invention:

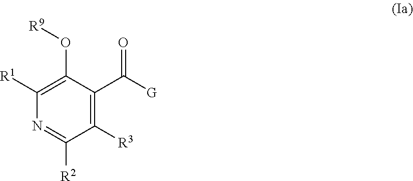

wherein G (comprising —N(H)—, R⁴, R⁵, R⁶, R⁷, R⁸ and L¹) according to compounds of Formula (I) of the invention is

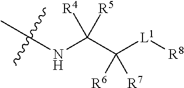

and wherein the R⁹ substituents are as defined below:

| | R9 |
|---|---|
| 1 | 3-cyanophenyl |
| 2 | 3-(difluoromethoxy)phenyl |
| 3 | 4-chlorophenyl |
| 4 | 4-fluorophenyl |
| 5 | 3-fluorophenyl |
| 6 | 3-methoxyphenyl |
| 7 | 4-methoxyphenyl |
| 8 | 3,4-dimethoxyphenyl |
| 9 | 4-tert-butoxyphenyl |
| 10 | 3,4-difluorophenyl |
| 11 | 3-ethylphenyl |
| 12 | 3-ethoxyphenyl |
| 13 | 3-acetamidophenyl |
| 14 | 4-cyanophenyl |
| 15 | 3,4-dichlorophenyl |
| 16 | 2,4-dichlorophenyl |
| 17 | 3,5-difluorophenyl |
| 18 | 3,5-dichlorophenyl |
| 19 | 2,4-difluorophenyl |
| 20 | 2-fluoro-3-(cyano)phenyl |
| 21 | 2-chlorophenyl |
| 22 | 2-fluorophenyl |
| 23 | 4-(trifluoromethyl)phenyl |
| 24 | 3-(trifluoromethoxy)phenyl |
| 25 | 4-(trifluoromethoxy)phenyl |
| 26 | 4-fluoro-3-methoxy-phenyl |
| 27 | 3-chloro-4-cyano-phenyl |
| 28 | 4-chloro-3-methyl-phenyl |
| 29 | 4-ethylphenyl |
| 30 | 3-chloro-4-methylsulfanyl-phenyl |
| 31 | 3-prop-2-ynoxyphenyl |
| 32 | 3-fluoro-4-methoxy-phenyl |
| 33 | 4-(cyanomethyl)phenyl |
| 34 | 3-methyl-4-methylsulfanyl-phenyl |
| 35 | 4-ethoxyphenyl |
| 36 | 4-chloro-3-ethoxy-phenyl |
| 37 | 4-fluoro-3-(trifluoromethyl)phenyl |
| 38 | 4-chloro-3-(trifluoromethyl)phenyl |
| 39 | 4-chloro-3-(trifluoromethoxy)phenyl |
| 40 | 2-fluoro-3-methylphenyl |
| 41 | 2-fluoro-3-(trifluoromethyl)phenyl |

The following compounds are thus specifically described in Tables B-1 to B-40 with the substituents for Formula (Ia) as follows:

Table B-1: This table provides 41 compounds B-1.01 and B-1.41 of formula (Ia) wherein R¹ is hydrogen, R² is methyl, R³ is hydrogen, G is [2-(2,4-dichlorophenyl)-2-fluoro-ethyl]amino] and R⁹ substituents are as defined in Table B above.
For example, compound B-1.05 and B-1.06 have the following structure;

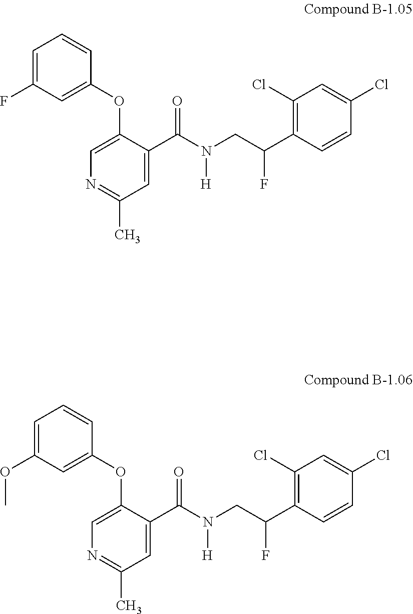 [SMILES: Cc1cc(C(=O)NCC(F)c2ccc(Cl)cc2Cl)c(Oc2cccc(F)c2)cn1 ; COc1cccc(Oc2cnc(C)cc2C(=O)NCC(F)c2ccc(Cl)cc2Cl)c1]

Table B-2: This table provides 41 compounds B-2.01 to B-2.41 of formula (Ia) wherein R¹ is hydrogen, R² is methyl, R³ is hydrogen, G is [2-(2,4-dichlorophenyl)ethylamino] and R⁹ substituents are as defined in Table B above.
Table B-3: This table provides 41 compounds B-3.01 to B-3.41 of formula (Ia) wherein R¹ is hydrogen, R² is methyl, R³ is hydrogen, G is [2-(2,4-dichlorophenyl)-2,2-difluoro-ethyl]amino] and R⁹ substituents are as defined in Table B above
Table B-4: This table provides 41 compounds B-4.01 to B-4.41 of formula (Ia) wherein R¹ is hydrogen, R² is chloro, R³ is hydrogen, G is [2-(2,4-dichlorophenyl)-2-fluoro-ethyl]amino] and R⁹ substituents are as defined in Table B above.
Table B-5: This table provides 41 compounds B-5.01 to B-5.41 of formula (Ia) wherein R¹ is hydrogen, R² is chloro, R³ is hydrogen, G is [2-(2,4-dichlorophenyl)-2,2-difluoro-ethyl]amino] and R⁹ substituents are as defined in Table B above.
Table B-6: This table provides 41 compounds B-6.01 to B-6.41 of formula (Ia) wherein R¹ is hydrogen, R² is chloro, R³ is hydrogen, G is [2-(2,4-dichlorophenyl)ethylamino] and R⁹ substituents are as defined in Table B above.
Table B-7: This table provides 41 compounds B-7.01 to B-7.41 of formula (Ia) wherein R¹ is hydrogen, R² is methyl, R³ is hydrogen, G is [2-(2,4-dimethylphenyl)-2-fluoro-ethyl]amino] and R⁹ substituents are as defined in Table B above.
Table B-8: This table provides 41 compounds B-8.01 to B-8.41 of formula (Ia) wherein R¹ is hydrogen, R² is methyl, R³ is hydrogen, G is [2-(2,4-dimethylphenyl)ethylamino] and R⁹ substituents are as defined in Table B above.
Table B-9: This table provides 41 compounds B-9.01 to B-9.41 of formula (Ia) wherein R¹ is hydrogen, R² is methyl, R³ is hydrogen, G is [2-(2,4-dimethylphenyl)-2,2-difluoro-ethyl]amino] and R⁹ substituents are as defined in Table B above.
Table B-10: This table provides 41 compounds B-10.01 to B-10.41 of formula (Ia) wherein R¹ is hydrogen, R² is chloro, R³ is hydrogen, G is [2-(2,4-dimethylphenyl)-2-fluoro-ethyl]amino] and R⁹ substituents are as defined in Table B above.
Table B-11: This table provides 41 compounds B-11.01 to B-11.41 of formula (Ia) wherein R¹ is hydrogen, R² is chloro, R³ is hydrogen, G is [2-(2,4-dimethylphenyl)ethylamino] and R⁹ substituents are as defined in Table B above.
Table B-12: This table provides 41 compounds B-12.01 to B-12.41 of formula (Ia) wherein R¹ is hydrogen, R² is chloro, R³ is hydrogen, G is [2-(2,4-dimethylphenyl)-2,2-difluoro-ethyl]amino] and R⁹ substituents are as defined in Table B above.
Table B-13: This table provides 41 compounds B-13.01 to B-13.41 of formula (Ia) wherein R¹ is hydrogen, R² is hydrogen, R³ is fluoro, G is [2-(2,4-dichlorophenyl)-2-fluoro-ethyl]amino] and R⁹ substituents are as defined in Table B above.
Table B-14: This table provides 41 compounds B-14.01 to B-14.41 of formula (Ia) wherein R¹ is hydrogen, R² is hydrogen, R³ is fluoro, G is [2-(2,4-dichlorophenyl)ethylamino] and R⁹ substituents are as defined in Table B above.
Table B-15: This table provides 41 compounds B-15.01 to B-15.41 of formula (Ia) wherein R¹ is hydrogen, R² is hydrogen, R³ is fluoro, G is [2-(2,4-dichlorophenyl)-2,2-difluoro-ethyl]amino] and R⁹ substituents are as defined in Table B above.
Table B-16: This table provides 41 compounds B-16.01 to B-16.41 of formula (Ia) wherein R¹ is hydrogen, R² is hydrogen, R³ is methyl, G is [2-(2,4-dichlorophenyl)-2-fluoro-ethyl]amino] and R⁹ substituents are as defined in Table B above.
Table B-17: This table provides 41 compounds B-17.01 to B-17.41 of formula (Ia) wherein R¹ is hydrogen, R² is hydrogen, R³ is methyl, G is [2-(2,4-dichlorophenyl)ethylamino] and R⁹ substituents are as defined in Table B above.
Table B-18: This table provides 41 compounds B-18.01 to B-18.41 of formula (Ia) wherein R¹ is hydrogen, R² is hydrogen, R³ is methyl, G is [2-(2,4-dichlorophenyl)-2,2-difluoro-ethyl]amino] and R⁹ substituents are as defined in Table B above.
Table B-19: This table provides 41 compounds B-19.01 to B-19.41 of formula (Ia) wherein R¹ is hydrogen, R² is hydrogen, R³ is fluoro, G is [2-(2,4-dimethylphenyl)-2-fluoro-ethyl]amino] and R⁹ substituents are as defined in Table B above.
Table B-20: This table provides 41 compounds B-20.01 to B-20.41 of formula (Ia) wherein R¹ is hydrogen, R² is hydrogen, R³ is fluoro, G is [2-(2,4-dimethylphenyl)ethylamino] and R⁹ substituents are as defined in Table B above.
Table B-21: This table provides 41 compounds B-21.01 to B-21.41 of formula (Ia) wherein R¹ is hydrogen, R² is hydrogen, R³ is fluoro, G is [2-(2,4-dimethylphenyl)-2,2-difluoro-ethyl]amino] and R⁹ substituents are as defined in Table B above.
Table B-22: This table provides 41 compounds B-22.01 to B-22.41 of formula (Ia) wherein R¹ is hydrogen, R² is hydrogen, R³ is methyl, G is [2-(2,4-dimethylphenyl)-2-fluoro-ethyl]amino] and R⁹ substituents are as defined in Table B above.
Table B-23: This table provides 41 compounds B-23.01 to B-23.41 of formula (Ia) wherein R¹ is hydrogen, R² is hydrogen, R³ is methyl, G is [2-(2,4-dimethylphenyl)ethylamino] and R⁹ substituents are as defined in Table B above.
Table B-24: This table provides 41 compounds B-24.01 to B-24.41 of formula (Ia) wherein R¹ is hydrogen, R² is hydrogen, R³ is methyl, G is [2-(2,4-dimethylphenyl)-2,2-difluoro-ethyl]amino] and R⁹ substituents are as defined in Table B above.
Table B-25: This table provides 41 compounds B-25.01 to B-25.41 of formula (Ia) wherein R¹ is hydrogen, R² is hydrogen, R³ is methoxy, G is [2-(2,4-dichlorophenyl)-2-fluoro-ethyl]amino] and R⁹ substituents are as defined in Table B above.
Table B-26: This table provides 41 compounds B-26.01 to B-26.41 of formula (Ia) wherein R¹ is hydrogen, R² is hydrogen, R³ is methoxy, G is [2-(2,4-dichlorophenyl)ethylamino] and R⁹ substituents are as defined in Table B above.
Table B-27: This table provides 41 compounds B-27.01 to B-27.41 of formula (Ia) wherein R¹ is hydrogen, R² is hydrogen, R³ is methoxy, G is [2-(2,4-dichlorophenyl)-2,2-difluoro-ethyl]amino] and R⁹ substituents are as defined in Table B above.
Table B-28: This table provides 41 compounds B-28.01 to B-28.41 of formula (Ia) wherein R¹ is hydrogen, R² is hydrogen, R³ is methoxy, G is [2-(2,4-dimethylphenyl)-2-fluoro-ethyl]amino] and R⁹ substituents are as defined in Table B above.
Table B-29: This table provides 41 compounds B-29.01 to B-29.41 of formula (Ia) wherein R¹ is hydrogen, R² is hydrogen, R³ is methoxy, G is [2-(2,4-dimethylphenyl)ethylamino] and R⁹ substituents are as defined in Table B above.
Table B-30: This table provides 41 compounds B-30.01 to B-30.41 of formula (Ia) wherein R¹ is hydrogen, R² is hydrogen, R³ is methoxy, G is [[2-(2,4-dimethylphenyl)-2,2-difluoro-ethyl]amino] and R⁹ substituents are as defined in Table B above.
Table B-31: This table provides 41 compounds B-31.01 and B-31.41 of formula (Ia) wherein R¹ is hydrogen, R² is methyl, R³ is hydrogen, G is [(2S)-2-(2,4-dichlorophenyl)-2-fluoro-ethyl]amino] and R⁹ substituents are as defined in Table B above.
Table B-32: This table provides 41 compounds B-32.01 to B-32.41 of formula (Ia) wherein R¹ is hydrogen, R² is methyl, R³ is hydrogen, G is [(2S)-2-(2,4-dimethylphenyl)-2-fluoro-ethyl]amino] and R⁹ substituents are as defined in Table B above.
Table B-33: This table provides 41 compounds B-33.01 to B-33.41 of formula (Ia) wherein R¹ is hydrogen, R² is chloro, R³ is hydrogen, G is [(2S)-2-(2,4-dimethylphenyl)-2-fluoro-ethyl]amino] and R⁹ substituents are as defined in Table B above.
Table B-34: This table provides 41 compounds B-34.01 to B-34.41 of formula (Ia) wherein R¹ is hydrogen, R² is chloro, R³ is hydrogen, G is [(2S)-2-(2,4-dichlorophenyl)-2-fluoro-ethyl]amino] and R⁹ substituents are as defined in Table B above.
Table B-35: This table provides 41 compounds B-35.01 to B-35.41 of formula (Ia) wherein R¹ is hydrogen, R² is hydrogen, R³ is methyl, G is [(2S)-2-(2,4-dichlorophenyl)-2-fluoro-ethyl]amino] and R⁹ substituents are as defined in Table B above.
Table B-36: This table provides 41 compounds B-36.01 to B-36.41 of formula (Ia) wherein R¹ is hydrogen, R² is hydrogen, R³ is methyl, G is [(2S)-2-(2,4-dimethylphenyl)-2-fluoro-ethyl]amino] and R⁹ substituents are as defined in Table B above.
Table B-37: This table provides 41 compounds B-37.01 to B-37.41 of formula (Ia) wherein R¹ is hydrogen, R² is hydrogen, R³ is fluoro, G is [(2S)-2-(2,4-dichlorophenyl)-2-fluoro-ethyl]amino] and R⁹ substituents are as defined in Table B above.
Table B-38: This table provides 41 compounds B-38.01 to B-38.41 of formula (Ia) wherein R¹ is hydrogen, R² is hydrogen, R³ is fluoro, G is [(2S)-2-(2,4-dimethylphenyl)-2-fluoro-ethyl]amino] and R⁹ substituents are as defined in Table B above.
Table B-39: This table provides 41 compounds B-39.01 to B-39.41 of formula (Ia) wherein R¹ is hydrogen, R² is hydrogen, R³ is methoxy, G is [(2S)-2-(2,4-dichlorophenyl)-2-fluoro-ethyl]amino] and R⁹ substituents are as defined in Table B above.
Table B-40: This table provides 41 compounds B-40.01 to B-40.41 of formula (Ia) wherein R¹ is hydrogen, R² is hydrogen, R³ is methoxy, G is [(2S)-2-(2,4-dimethylphenyl)-2-fluoro-ethyl]amino] and R⁹ substituents are as defined in Table B above.

Preferably, the compound of Formula (I) does not represent compounds A-01.61, A-01.62, A-02.61, A-02.62, A-03.61, A-03.62, A-05.23, A-05.61, A-05.62, A-06.23, A-06.61, A-06.62, A-07.23, A-07.59, A-07.61, A-07.62, A-08.62, A-27.61, A-27.62, B-08.06, B-09.06, B-11.02, B-11.06, B-11.11, B-11.12, B-11.15, B-11.23, B-11.24, B-11.25, B-11.38, B-11.40, or B-12.06. Also preferably, the compound of Formula (I) does not represent compounds 1.3, 1.16, 1.41, 1.42, 1.60, 1.61, 1.70, 1.76 or 1.77 as listed in Table T1.

### EXAMPLES

The Examples which follow serve to illustrate the invention.

The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Compounds of Formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius (° C.) and “mp.” means melting point. LC/MS means Liquid Chromatography Mass Spectrometry and the description of the apparatus and the method A is as follows:

### Method A

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 L/h, Mass range: 100 to 900 Da) and an Acquity UPLC from Waters: Binary pump, heated column compartment , diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (mL/min) 0.85

### Method B

Spectra were recorded on a Mass Spectrometer from Waters (Acquity QDa Mass Spectrometer) equipped with an electrospray source (Polarity: Positive and Negative Polarity Switch), Capillary: 0.8 kV, Cone range: 25 V, Source Temperature: 120° C., Desolvation Temperature: 600° C., Cone Gas Flow: 50 I/h, Desolvation Gas Flow: 1000 L/h, Mass range: 110 to 850 Da and an an Acquity UPLC from Waters: Quaternary solvent manager, heated column compartment, diode-array detector and ELSD detector. Column: Waters UPLC HSS T3, 1.8 μm, 30×2.1 mm, Temp: 40° C., PDA Wavelength range (nm): 230 to 400, Solvent Gradient: A=Water with 0.1% formic acid: Acetonitrile: 95: 5 v/v, B=Acetonitrile with 0.05% formic acid, : Gradient: 0 min-1.0 min , 10% B-90% A; 1.0 min-4.50 min 10% -100% B; 4.51 min-5.30 min , 100% B, 0% A; 5.31 min-5.50 min 100% -10% B; 5.51 min-6.00 min ,10% B, 90% A; Flow (ml/min) 0.6.

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

### Emulsifiable Concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

### Extruded Granules

| | |
|---|---|
| Active ingredient [compound of formula (I)] | 15% |
| sodium lignosulfonate | 2% |
| Carboxymethylcellulose | 1% |
| Kaolin | 82% |

The active ingredient is mixed and ground with the adjuvants, and the mixture is moistened with water. The mixture is extruded and then dried in a stream of air.

### Coated Granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

### Suspension Concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| Carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow-Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### LIST OF ABBREVIATIONS

aq.=aqueous
° C.=degrees Celsius
DCM=dichloromethane
DMF=dimethylformamide
DMSO=dimethyl sulfoxide
DMSO-d₆=deuterated dimethyl sulfoxide
EtOAc=ethyl acetate
equiv.=equivalent
h=hour(s)
M=molar
min=minutes
mp=melting point
ppm=parts per million
RT=room temperature
Rₜ=retention time
LC/MS=Liquid Chromatography Mass Spectrometry (description of the apparatus and the methods used for LC/MS analysis are given above)

### Preparation Examples

The following examples further illustrate, but do not limit, the invention. Those skilled in the art will promptly recognize appropriate variations from the procedures both as to reactants and as to reaction conditions and techniques.

¹H NMR spectra are recorded at 400 MHz (megahertz) unless indicated otherwise and chemical shifts are recorded in ppm. The following abbreviations are used: s=singlet; br s=broad singlet; d=doublet; dd=double doublet; dt=double triplet; t=triplet, tt=triple triplet, q=quartet, quin=quintuplet, sept=septet; m=multiplet.

Example 1: This example illustrates the preparation of 2-chloro-N-[2-(2,4-dichlorophenyl)ethyl]-5-(3-methylphenoxy)pyridine-4-carboxamide (Compound 1.4 of Table T1).

### a) Preparation of 5-bromo-2-chloro-N-[2-(2,4-dichlorophenyl)ethyl]pyridine-4-carboxamide

A mixture of 5-bromo-2-chloropyridine-4-carboxylic acid (0.52 g, 2.19 mmol), 2-(2,4-dichlorophenyl)ethylamine (0.5 g, 2.63 mmol), propanephosphonic acid anhydride (T3P, 50% in ethyl acetate, 4.19 g, 6.59 mmol) and triethylamine (0.48 g, 4.8 mmol) in 11 mL of ethyl acetate was stirred for 2 h at room temperature. Subsequently, the reaction mixture was diluted with ethyl acetate and washed with saturated aqueous sodium bicarbonate solution and brine. The organic layer was dried over sodium sulfate and evaporated under reduced pressure, the residue was purified by chromatography on silica gel, using cyclohexane/ethyl acetate as eluent system to yield 5-bromo-2-chloro-N-[2-(2,4-dichlorophenyl)ethyl]pyridine-4-carboxamide (0.78 g, 1.91 mmol).

¹H NMR (400 MHz, CDCl₃) δ ppm 3.12 (t, 2H), 3.78 (t, 2H), 6.09 (bs, 1H), 7.25 (d, 1H), 7.43-7.50 (m, 2H), 8.58 (s, 1H), 9.11 (s, 1H).

### b) Preparation of 2-chloro-N-[2-(2,4-dichlorophenyl)ethyl]-5-(3-methylphenoxy)pyridine-4-carboxamide

N-Benzyl-N′-(2-methyl-1-naphthyl)oxamide (10 mg, 0.03 mmol), potassium phosphate (0.13 g, 0.63 mmol) and copper (I) iodide (3.1 mg, 0.02 mmol) were added to a solution of 5-bromo-2-chloro-N-[2-(2,4-dichlorophenyl)ethyl]pyridine-4-carboxamide (0.13 g, 0.32 mmol) and m-cresol (42 mg, 0.38 mmol) in 1 mL of dimethylsulfoxide. The reaction mixture was stirred for 30 min at 110° C., then cooled to room temperature, diluted with water and extracted with ethyl acetate. The organic layer was washed with brine, dried over sodium sulfate and concentrated under reduced pressure. The residue was purified by chromatography on silica gel, using cyclohexane/ethyl acetate as eluent system to yield 2-chloro-N-[2-(2,4-dichlorophenyl)ethyl]-5-(3-methylphenoxy)pyridine-4-carboxamide (94 mg, 0.21 mmol).

¹H NMR (400 MHz, CDCl₃) δ ppm 2.41 (s, 3H), 3.03 (t, 2H), 3.78 (t, 2H), 6.77-6.80 (m, 2H), 6.95 (d, 1H), 7.07-7.16 (m, 2H), 7.32 (t, 1H), 7.61 (bs, 1H), 7.98 (s, 1H), 8.10 (s, 1H).

Example 2: This example illustrates the preparation of N-[2-(2,4-dichlorophenyl)-2-fluoro-ethyl]-2-methyl-5-(3-methylphenoxy)pyridine-4-carboxamide (Compound 1.1 of Table T1).

### a) Preparation of 2-methyl-5-(3-methylphenoxy)pyridine-4-carboxylic acid

N-Benzyl-N′-(2-methyl-1-naphthyl)oxamide (37 mg, 0.11 mmol), potassium phosphate (0.50 g, 2.37 mmol) and copper (I) iodide (11 mg, 0.06 mmol) were added to a solution of ethyl 5-chloro-2-methylpyridine-4-carboxylate (0.22 g, 1.18 mmol) and m-cresol (0.13 g, 1.18 mmol) in 3 mL of dimethylsulfoxide. The reaction mixture was stirred for 1 h at 110° C., then cooled to room temperature, diluted with water and extracted with ethyl acetate. The organic layer was washed with brine, dried over sodium sulfate and concentrated under reduced pressure. The residue was purified by chromatography on silica gel, using cyclohexane/ethyl acetate as eluent system to yield 2-methyl-5-(3-methylphenoxy)pyridine-4-carboxylic acid (75 mg, 0.31 mmol).

¹H NMR (400 MHz, DMSO-d6) δ ppm 2.50-2.54 (m, 6H), 6.53-6.61 (m, 2H), 7.01-7.07 (m, 2H), 7.78 (s, 1H), 8.02 (s, 1H).

### b) Preparation of N-[2-(2,4-dichlorophenyl)-2-fluoro-ethyl]-2-methyl-5-(3-methylphenoxy)pyridine-4-carboxamide

A mixture of 2-methyl-5-(3-methylphenoxy)pyridine-4-carboxylic acid (25 mg, 0.1 mmol), 2-(2,4-dichlorophenyl)-2-fluoroethylamine hydrochloride (30 mg, 0.12 mmol), propanephosphonic acid anhydride (T3P, 50% in ethyl acetate, 0.19 g, 0.31 mmol) and triethylamine (24 mg, 0.24 mmol) in 1 mL of ethyl acetate was stirred for 2 h at room temperature. Subsequently, the reaction mixture was diluted with ethyl acetate and washed with saturated aqueous sodium bicarbonate solution and brine. The organic layer was dried over sodium sulfate and evaporated under reduced pressure, the residue was purified by chromatography on silica gel, using cyclohexane/ethyl acetate as eluent system to yield N-[2-(2,4-dichlorophenyl)-2-fluoro-ethyl]-2-methyl-5-(3-methylphenoxy)pyridine-4-carboxamide (23 mg, 0.05 mmol).

¹H NMR (400 MHz, CDCl₃) δ ppm 2.40 (s, 3H), 2.61 (s, 3H), 3.75-3.88 (m, 1H), 4.09-4.18 (m, 2H), 6.82-6.85 (m, 2H), 7.06-7.13 (m, 2H), 7.31-7.38 (m, 2H), 7.89 (s, 1H), 7.95 (bs, 1H), 8.18 (s, 1H).

TABLE T1
LCMS data and/or retention times (Rₜ) for compounds according to formula (I):

| | Compound | | Rₜ | | |
|---|---|---|---|---|---|
| Entry | name | Structure | (min) | Mass charge | Method |
| 1.1 | N-[2-(2,4- dichlorophenyl)- 2-fluoro-ethyl]-2- methyl-5-(3- methylphenoxy) pyridine-4- carboxamide | 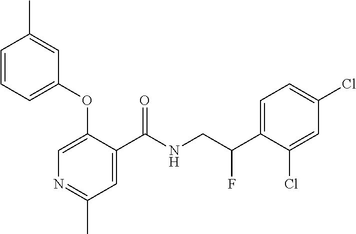 [SMILES: Cc1cccc(Oc2cnc(C)cc2C(=O)NCC(F)c2ccc(Cl)cc2Cl)c1] | 1.16 | 433 | A |
| 1.2 | N-[2-(2,4- dichlorophenyl) ethyl]-2-methyl-5- (3- methylphenoxy) pyridine-4- carboxamide | 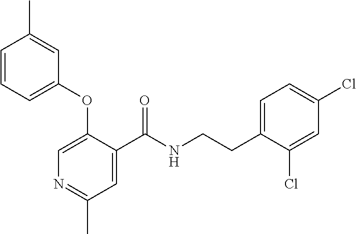 [SMILES: Cc1cccc(Oc2cnc(C)cc2C(=O)NCCc2ccc(Cl)cc2Cl)c1] | 1.16 | 415 | A |
| 1.3 | 2-chloro-N-[2- (2,4- dichlorophenyl)- 2-fluoro-ethyl]-5- (3- methylphenoxy) pyridine-4- carboxamide | 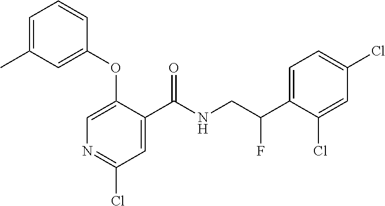 [SMILES: Cc1cccc(Oc2cnc(Cl)cc2C(=O)NCC(F)c2ccc(Cl)cc2Cl)c1] | 1.22 | 452 | A |
| 1.4 | 2-chloro-N-[2- (2,4- dichlorophenyl) ethyl]-5-(3- methylphenoxy) pyridine-4- carboxamide | 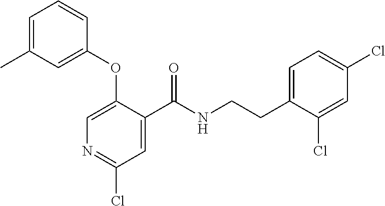 [SMILES: Cc1cccc(Oc2cnc(Cl)cc2C(=O)NCCc2ccc(Cl)cc2Cl)c1] | 1.24 | 435 | A |
| 1.5 | 2-bromo-5-(3- cyclopropyl- phenoxy)-N-[2-(2,4- dichlorophenyl)- 2-fluoro- ethyl]pyridine-4- carboxamide | 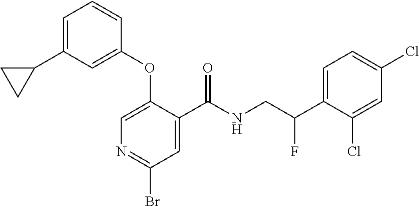 [SMILES: O=C(NCC(F)c1ccc(Cl)cc1Cl)c1cc(Br)ncc1Oc1cccc(C2CC2)c1] | 1.24 | 524 | A |
| 1.6 | 3-(3- cyclopropyl- phenoxy)-N-[2-(2,4- dichlorophenyl)- 2-fluoro-ethyl]-5- fluoro-pyridine-4- carboxamide | 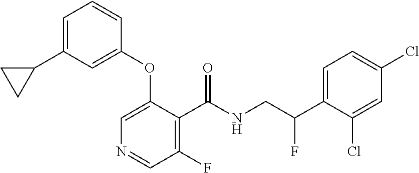 [SMILES: O=C(NCC(F)c1ccc(Cl)cc1Cl)c1c(F)cncc1Oc1cccc(C2CC2)c1] | 1.18 | 463 | A |
| 1.7 | 3-(3- cyclopropyl- phenoxy)-N-[2-(2,4- dichlorophenyl)- 2-fluoro-ethyl]-5- methyl-pyridine- 4-carboxamide | 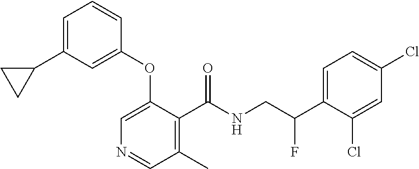 [SMILES: Cc1cncc(Oc2cccc(C3CC3)c2)c1C(=O)NCC(F)c1ccc(Cl)cc1Cl] | 1.15 | 458 | A |
| 1.8 | 3-bromo-5-(3- cyclopropyl- phenoxy)-N-[2-(2,4- dichlorophenyl)- 2-fluoro- ethyl]pyridine-4- carboxamide | 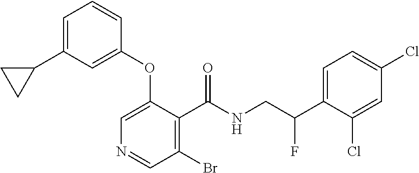 [SMILES: O=C(NCC(F)c1ccc(Cl)cc1Cl)c1c(Br)cncc1Oc1cccc(C2CC2)c1] | 1.19 | 524 | A |
| 1.9 | 5-(3- cyclopropyl- phenoxy)-N-[2-(2,4- dichlorophenyl)- 2-fluoro-ethyl]-2- (trifluoromethyl) pyridine-4- carboxamide | 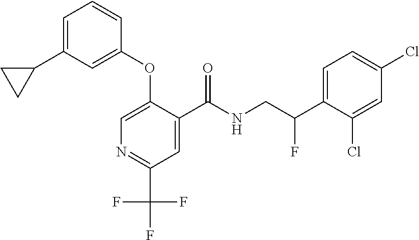 [SMILES: O=C(NCC(F)c1ccc(Cl)cc1Cl)c1cc(C(F)(F)F)ncc1Oc1cccc(C2CC2)c1] | 1.31 | 513 | A |
| 1.10 | 5-(3- cyclopropyl- phenoxy)-N-[2-(2,4- dichlorophenyl)- 2-fluoro-ethyl]-2- methylsulfonyl- pyridine-4- carboxamide | 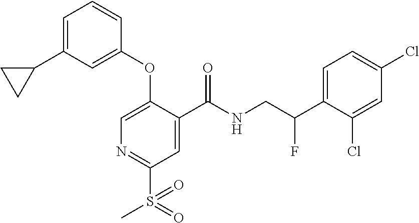 [SMILES: CS(=O)(=O)c1cc(C(=O)NCC(F)c2ccc(Cl)cc2Cl)c(Oc2cccc(C3CC3)c2)cn1] | 1.16 | 523 | A |
| 1.11 | 3-(3- cyclopropyl- phenoxy)-N-[2-(2,4- dichlorophenyl)- 2-fluoro- ethyl]pyridine-4- carboxamide | 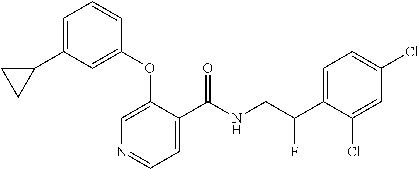 [SMILES: O=C(NCC(F)c1ccc(Cl)cc1Cl)c1ccncc1Oc1cccc(C2CC2)c1] | 1.19 | 447 | A |
| 1.12 | 3-(3- cyclopropyl- phenoxy)- N-[2-(2,4- dichlorophenyl)- 2-fluoro-ethyl]-2- methyl-pyridine- 4-carboxamide | 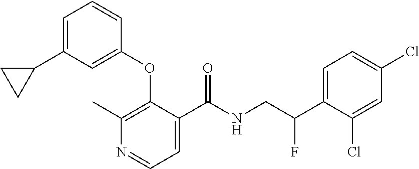 [SMILES: Cc1nccc(C(=O)NCC(F)c2ccc(Cl)cc2Cl)c1Oc1cccc(C2CC2)c1] | 1.16 | 459 | A |
| 1.13 | 5-(3- cyclopropyl- phenoxy)-N-[2-(2,4- dichlorophenyl) ethyl]-2-methyl- pyridine-4- carboxamide | 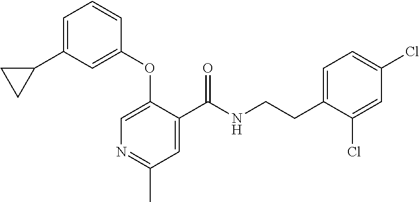 [SMILES: Cc1cc(C(=O)NCCc2ccc(Cl)cc2Cl)c(Oc2cccc(C3CC3)c2)cn1] | 1.20 | 441 | A |
| 1.14 | 5-(3- cyclopropyl- phenoxy)-N-[2-(2,4- dichlorophenyl)- 2-fluoro-ethyl]-2- methyl-pyridine- 4-carboxamide | 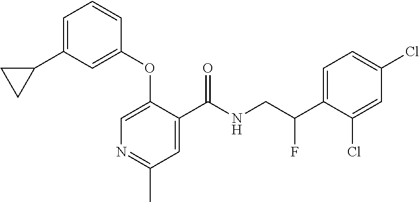 [SMILES: Cc1cc(C(=O)NCC(F)c2ccc(Cl)cc2Cl)c(Oc2cccc(C3CC3)c2)cn1] | 1.13 | 459 | A |
| 1.15 | 2-chloro-5-(3- cyclopropyl- phenoxy)-N-[2-(2,4- dichlorophenyl) ethyl]pyridine-4- carboxamide | 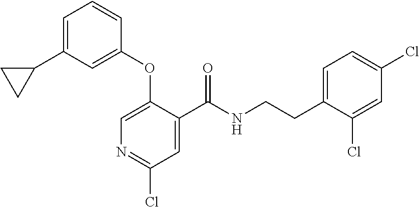 [SMILES: O=C(NCCc1ccc(Cl)cc1Cl)c1cc(Cl)ncc1Oc1cccc(C2CC2)c1] | 1.30 | 461 | A |
| 1.16 | 2-chloro-5-(3- cyclopropyl- phenoxy)-N-[2-(2,4- dichlorophenyl)- 2-fluoro- ethyl]pyridine-4- carboxamide | 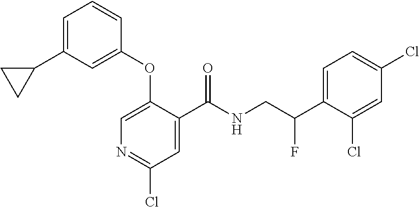 [SMILES: O=C(NCC(F)c1ccc(Cl)cc1Cl)c1cc(Cl)ncc1Oc1cccc(C2CC2)c1] | 1.39 | 478 | A |
| 1.17 | 2-chloro-3-(3- cyclopropyl- phenoxy)-N-[2-(2,4- dichlorophenyl)- 2-fluoro-ethyl]-6- methyl-pyridine- carboxamide | 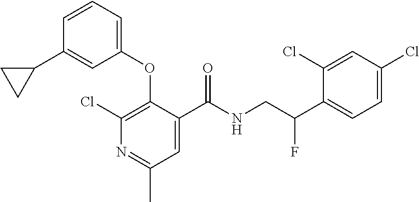 [SMILES: Cc1cc(C(=O)NCC(F)c2ccc(Cl)cc2Cl)c(Oc2cccc(C3CC3)c2)c(Cl)n1] | 1.27 | 492 | A |
| 1.18 | 6-chloro-3-(3- cyclopropyl- phenoxy)-N-[2-(2,4- dichlorophenyl)- 2-fluoro-ethyl]-2- methyl-pyridine- carboxamide | 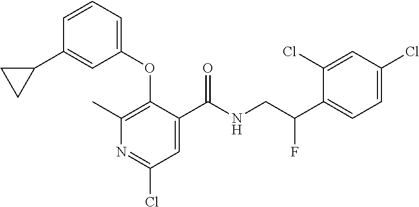 [SMILES: Cc1nc(Cl)cc(C(=O)NCC(F)c2ccc(Cl)cc2Cl)c1Oc1cccc(C2CC2)c1] | 1.30 | 492 | A |
| 1.19 | 5-(3- cyclopropyl- phenoxy)-N-[2-(2,4- dichlorophenyl) ethyl]-2- (trifluoromethyl) pyridine-4- carboxamide | 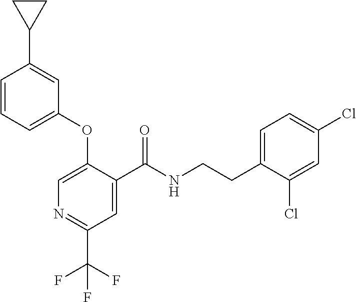 [SMILES: O=C(NCCc1ccc(Cl)cc1Cl)c1cc(C(F)(F)F)ncc1Oc1cccc(C2CC2)c1] | 1.33 | 494 | A |
| 1.20 | 2-cyano-5-(3- cyclopropyl- phenoxy)-N-[2-(2,4- dichlorophenyl) 2-fluoro- ethyl]pyridine-4- carboxamide | 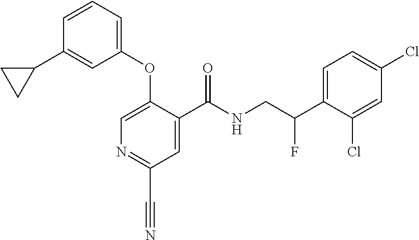 [SMILES: N#Cc1cc(C(=O)NCC(F)c2ccc(Cl)cc2Cl)c(Oc2cccc(C3CC3)c2)cn1] | 1.23 | 469 | A |
| 1.21 | 3-chloro-5-(3- cyclopropyl- phenoxy)-N-[2-(2,4- dichlorophenyl) ethyl]pyridine-4- carboxamide | 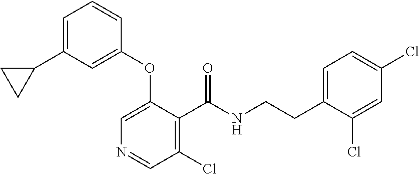 [SMILES: O=C(NCCc1ccc(Cl)cc1Cl)c1c(Cl)cncc1Oc1cccc(C2CC2)c1] | 1.18 | 463 | A |
| 1.22 | 2-cyclopropyl-5- (3- cyclopropyl- phenoxy)-N-[2-(2,4- dichlorophenyl)- 2-fluoro- ethyl]pyridine-4- carboxamide | 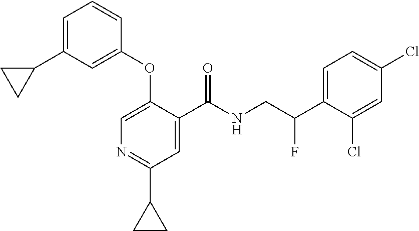 [SMILES: O=C(NCC(F)c1ccc(Cl)cc1Cl)c1cc(C2CC2)ncc1Oc1cccc(C2CC2)c1] | 1.32 | 485 | A |
| 1.23 | 2-chloro-N-[2-(2- cyanophenyl) ethyl]-5-(3- cyclopropyl- phenoxy)pyridine- 4-carboxamide | 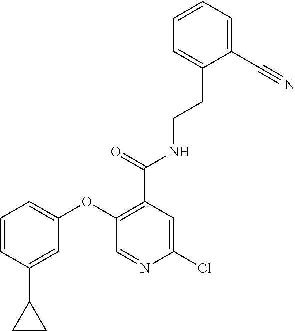 [SMILES: N#Cc1ccccc1CCNC(=O)c1cc(Cl)ncc1Oc1cccc(C2CC2)c1] | 2.14 | 4.17 | B |
| 1.24 | 2-chloro-N-[2-(4- chlorophenyl) ethyl]-5-(3- cyclopropyl- phenoxy)pyridine- 4-carboxamide | 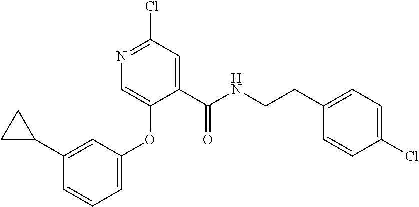 [SMILES: O=C(NCCc1ccc(Cl)cc1)c1cc(Cl)ncc1Oc1cccc(C2CC2)c1] | 1.97 | 426 | B |
| 1.25 | 2-chloro-N-[3-(4- chlorophenyl) propyl]-5-(3- cyclopropyl- phenoxy)pyridine-4- carboxamide | 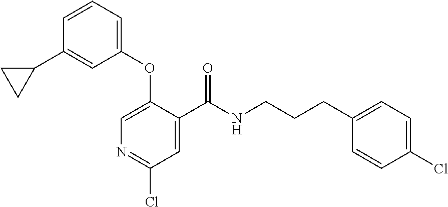 [SMILES: O=C(NCCCc1ccc(Cl)cc1)c1cc(Cl)ncc1Oc1cccc(C2CC2)c1] | 2.01 | 440 | B |
| 1.26 | 2-chloro-5-(3- cyclopropyl- phenoxy)-N-[2-(3,4- dimethoxyphenyl) ethyl]pyridine-4- carboxamide | 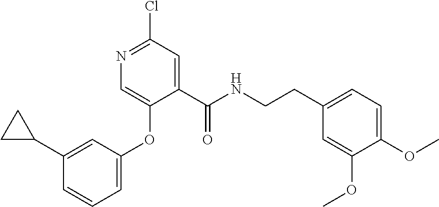 [SMILES: COc1ccc(CCNC(=O)c2cc(Cl)ncc2Oc2cccc(C3CC3)c2)cc1OC] | 1.73 | 452 | B |
| 1.27 | 2-chloro-5-(3- cyclopropyl- phenoxy)-N-[2-(2,3- dichlorophenyl) ethyl]pyridine-4- carboxamide | 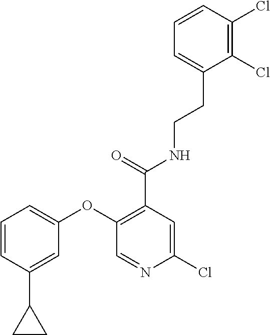 [SMILES: O=C(NCCc1cccc(Cl)c1Cl)c1cc(Cl)ncc1Oc1cccc(C2CC2)c1] | 2.05 | 460 | B |
| 1.28 | 2-chloro-5-(3- cyclopropyl- phenoxy)-N-[[1-(4- fluorophenyl) cyclobutyl]methyl] pyridine-4-carboxamide | 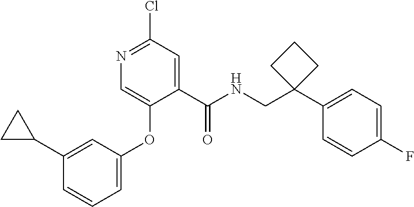 [SMILES: O=C(NCC1(c2ccc(F)cc2)CCC1)c1cc(Cl)ncc1Oc1cccc(C2CC2)c1] | 1.84 | 450 | B |
| 1.29 | 2-chloro-5-(3- cyclopropyl- phenoxy)-N-(2- phenylethyl) pyridine-4-carboxamide | 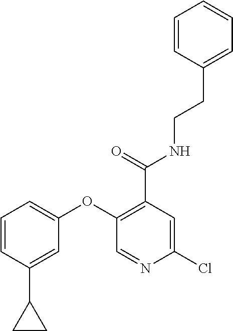 [SMILES: O=C(NCCc1ccccc1)c1cc(Cl)ncc1Oc1cccc(C2CC2)c1] | 1.87 | 392 | B |
| 1.30 | 2-chloro-5-(3- cyclopropyl- phenoxy)-N-(3- phenylpropyl) pyridine-4-carboxamide | 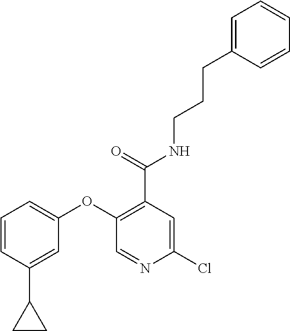 [SMILES: O=C(NCCCc1ccccc1)c1cc(Cl)ncc1Oc1cccc(C2CC2)c1] | 1.91 | 406 | B |
| 1.31 | 2-chloro-5-(3- cyclopropyl- phenoxy)-N-[2-(4- fluorophenyl)ethyl]pyridine- 4-carboxamide | 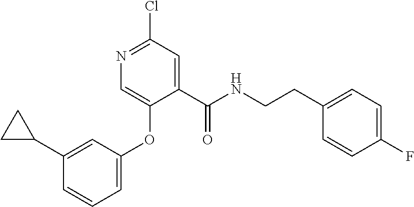 [SMILES: O=C(NCCc1ccc(F)cc1)c1cc(Cl)ncc1Oc1cccc(C2CC2)c1] | 1.86 | 410 | B |
| 1.32 | 2-chloro-5-(3- cyclopropyl- phenoxy)-N-[2-(2,4- difluorophenyl)- 2,2-difluoro- ethyl]pyridine-4- carboxamide | 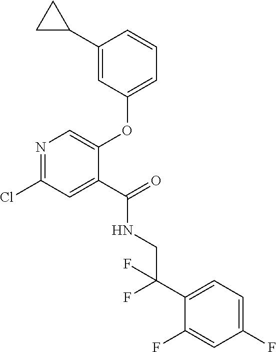 [SMILES: O=C(NCC(F)(F)c1ccc(F)cc1F)c1cc(Cl)ncc1Oc1cccc(C2CC2)c1] | 1.90 | 464 | B |
| 1.33 | 2-chloro-N-[2-(2- chlorophenyl) ethyl]-5-(3- cyclopropyl- phenoxy)pyridine-4- carboxamide | 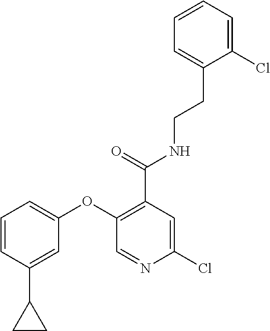 [SMILES: O=C(NCCc1ccccc1Cl)c1cc(Cl)ncc1Oc1cccc(C2CC2)c1] | 1.96 | 446 | B |
| 1.34 | 2-chloro-N-[2-(4- cyanophenyl) ethyl]-5-(3- cyclopropyl- phenoxy) pyridine-4- carboxamide | 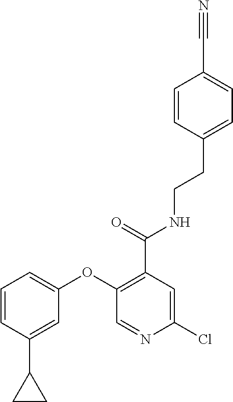 [SMILES: N#Cc1ccc(CCNC(=O)c2cc(Cl)ncc2Oc2cccc(C3CC3)c2)cc1] | 1.70 | 417 | B |
| 1.35 | 2-chloro-5-(3- cyclopropyl- phenoxy)-N-[2-(4- methoxyphenyl) ethyl]pyridine-4- carboxamide | 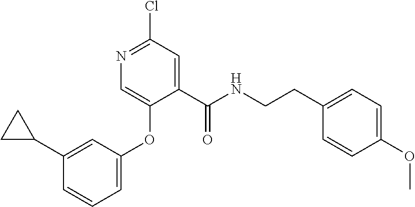 [SMILES: COc1ccc(CCNC(=O)c2cc(Cl)ncc2Oc2cccc(C3CC3)c2)cc1] | 1.83 | 422 | B |
| 1.36 | 2-chloro-5-(3- cyclopropyl- phenoxy)-N-[2-(o- tolyl)ethyl]pyridine- 4-carboxamide | 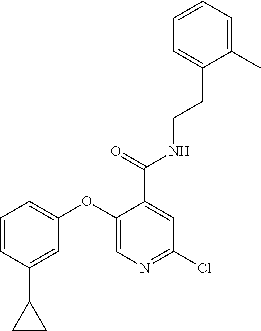 [SMILES: Cc1ccccc1CCNC(=O)c1cc(Cl)ncc1Oc1cccc(C2CC2)c1] | 1.76 | 406 | B |
| 1.37 | N-[2-(2,4- dimethylphenyl)- 2-fluoro-ethyl]-5- (3- ethylphenoxy)-2- methyl-pyridine- 4-carboxamide | 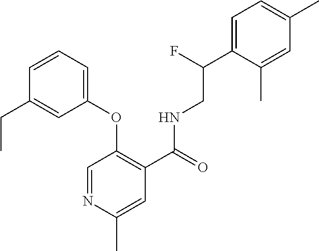 [SMILES: CCc1cccc(Oc2cnc(C)cc2C(=O)NCC(F)c2ccc(C)cc2C)c1] | 1.20 | 406 | A |
| 1.38 | N-[2-(2,4- dimethylphenyl)- 2-fluoro-ethyl]-5- (3- ethynylphenoxy)- 2-methyl- pyridine-4- carboxamide | 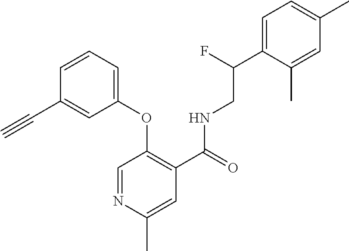 [SMILES: C#Cc1cccc(Oc2cnc(C)cc2C(=O)NCC(F)c2ccc(C)cc2C)c1] | 1.10 | 403 | A |
| 1.39 | 5-(3- cyclopropyl- phenoxy)-N-[2-(2,4- dimethylphenyl)- 2-fluoro-ethyl]-2- methyl-pyridine- 4-carboxamide | 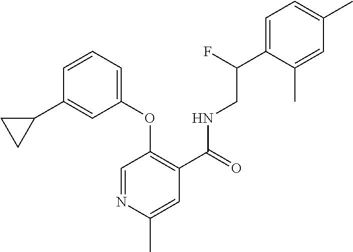 [SMILES: Cc1ccc(C(F)CNC(=O)c2cc(C)ncc2Oc2cccc(C3CC3)c2)c(C)c1] | 1.19 | 419 | A |
| 1.40 | 2-chloro-5-(3- cyclopropyl- phenoxy)-N-[2-(2,4- dichlorophenyl)- 2-fluoro- propyl]pyridine- 4-carboxamide | 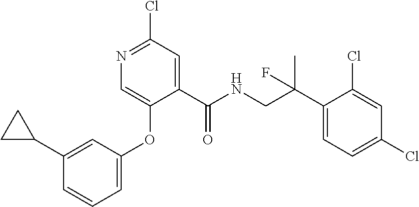 [SMILES: CC(F)(CNC(=O)c1cc(Cl)ncc1Oc1cccc(C2CC2)c1)c1ccc(Cl)cc1Cl] | 2.16 | 493 | B |
| 1.41 | 5-(3- cyclopropyl- phenoxy)-N-[(2S)-2- (2,4- dichlorophenyl)- 2-fluoro-ethyl]-2- methyl-pyridine- 4-carboxamide | 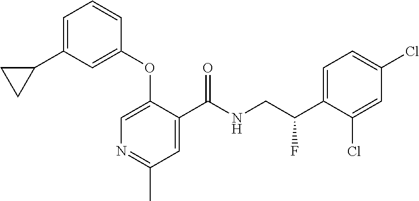 [SMILES: Cc1cc(C(=O)NC[C@@H](F)c2ccc(Cl)cc2Cl)c(Oc2cccc(C3CC3)c2)cn1] | 1.22 | 459 | A |
| 1.42 | 5-(3- cyclopropyl- phenoxy)-N-[(2R)-2- (2,4- dichlorophenyl)- 2-fluoro-ethyl]-2- methyl-pyridine- 4-carboxamide | 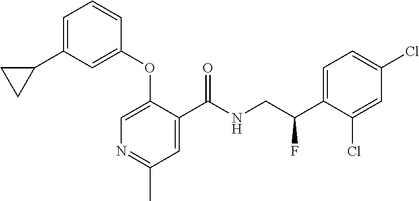 [SMILES: Cc1cc(C(=O)NC[C@H](F)c2ccc(Cl)cc2Cl)c(Oc2cccc(C3CC3)c2)cn1] | 1.22 | 459 | A |
| 1.43 | 2-bromo-N-[2- (2,4- dimethylphenyl)- 2-fluoro-ethyl]-5- (3- ethynylphenoxy) pyridine-4- carboxamide | 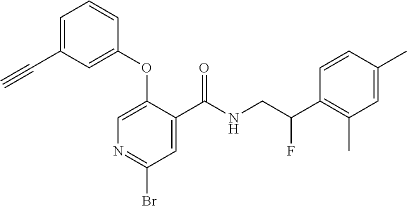 [SMILES: C#Cc1cccc(Oc2cnc(Br)cc2C(=O)NCC(F)c2ccc(C)cc2C)c1] | 1.20 | 466 | A |
| 1.44 | 2-chloro-N-[2- (3,4- dimethoxyphenyl) ethyl]-5- phenoxy- pyridine-4- carboxamide | 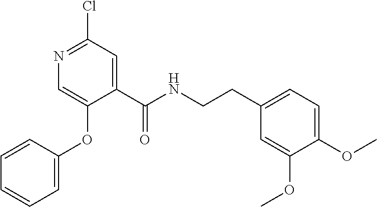 [SMILES: COc1ccc(CCNC(=O)c2cc(Cl)ncc2Oc2ccccc2)cc1OC] | 1.51 | 412 | B |
| 1.45 | 2-chloro-N-[2- (2,4- dichlorophenyl) ethyl]-5-phenoxy- pyridine-4- carboxamide | 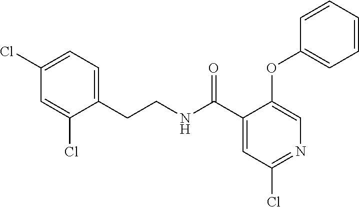 [SMILES: O=C(NCCc1ccc(Cl)cc1Cl)c1cc(Cl)ncc1Oc1ccccc1] | 1.90 | 420 | B |
| 1.46 | 2-chloro-N-[2-(3- chlorophenyl) ethyl]-5-(3- methylphenoxy) pyridine-4- carboxamide | 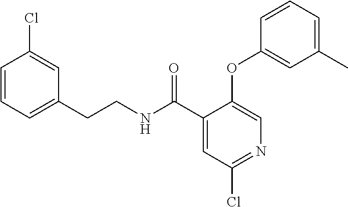 [SMILES: Cc1cccc(Oc2cnc(Cl)cc2C(=O)NCCc2cccc(Cl)c2)c1] | 1.68 | 400 | B |
| 1.47 | 2-chloro-N-[2-(4- chlorophenyl) ethyl]-5-(3- methylphenoxy) pyridine-4- carboxamide | 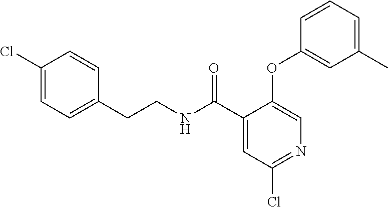 [SMILES: Cc1cccc(Oc2cnc(Cl)cc2C(=O)NCCc2ccc(Cl)cc2)c1] | 1.88 | 400 | B |
| 1.48 | 2-chloro-N-[2- (3,4- dimethoxyphenyl) ethyl]-5-(3- methylphenoxy) pyridine-4- carboxamide | 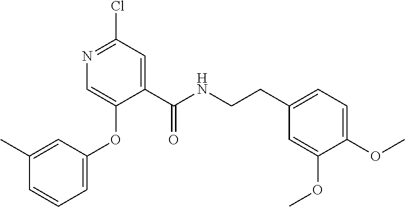 | 1.63 | 426 | B |
| 1.49 | N-(1- benzylcyclopropyl)- 2-chloro-5-(3- cyclopropylphenoxy) pyridine-4-carboxamide | 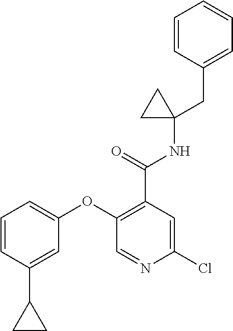 [SMILES: O=C(NC1(Cc2ccccc2)CC1)c1cc(Cl)ncc1Oc1cccc(C2CC2)c1] | 1.74 | 418 | B |
| 1.50 | 2-chloro-5-(3- cyclopropylphenoxy)- N-[2-(3,5- dimethylphenyl) ethyl]pyridine-4- carboxamide | 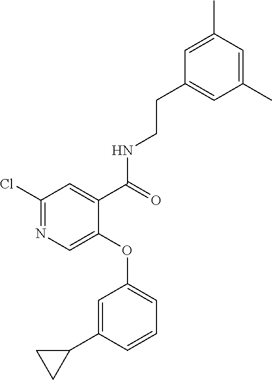 [SMILES: Cc1cc(C)cc(CCNC(=O)c2cc(Cl)ncc2Oc2cccc(C3CC3)c2)c1] | 1.87 | 420 | B |
| 1.51 | 2-chloro-N-[[1- (3,4- dichlorophenyl) cyclopropyl]methyl]- 5-(3- methylphenoxy) pyridine-4-carboxamide | 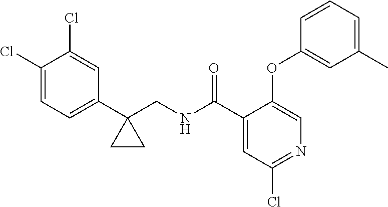 [SMILES: Cc1cccc(Oc2cnc(Cl)cc2C(=O)NCC2(c3ccc(Cl)c(Cl)c3)CC2)c1] | 209 | 460 | B |
| 1.52 | 2-chloro-N-[2-(4- fluorophenyl) ethyl]-5-(3- methylphenoxy) pyridine-4- carboxamide | 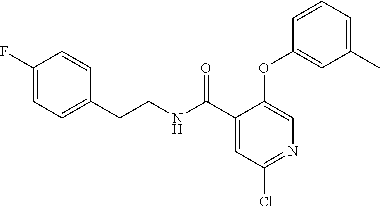 [SMILES: Cc1cccc(Oc2cnc(Cl)cc2C(=O)NCCc2ccc(F)cc2)c1] | 1.77 | 384 | B |
| 1.53 | 2-chloro-5-(3- cyclopropyl- phenoxy)-N-[2-(p- tolyl)ethyl]pyridine-4- carboxamide | 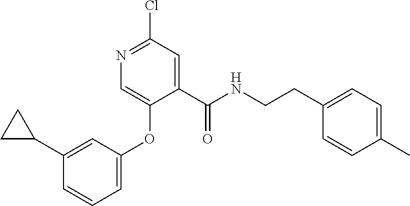 [SMILES: Cc1ccc(CCNC(=O)c2cc(Cl)ncc2Oc2cccc(C3CC3)c2)cc1] | 1.97 | 4.06 | B |
| 1.54 | 2-chloro-N-[2-(2- chlorophenyl) ethyl]-5-(3- methylphenoxy) pyridine-4- carboxamide | 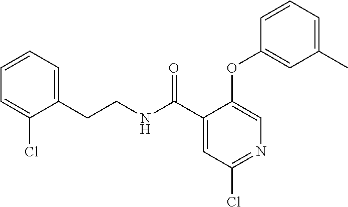 [SMILES: Cc1cccc(Oc2cnc(Cl)cc2C(=O)NCCc2ccccc2Cl)c1] | 1.87 | 400 | B |
| 1.55 | 2-chloro-N-[2-(4- methoxyphenyl) ethyl]-5-(3- methylphenoxy) pyridine-4- carboxamide | 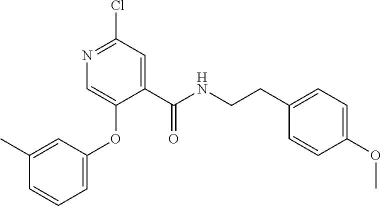 | 1.74 | 396 | B |
| 1.56 | 2-chloro-5-(3- methylphenoxy)- N-[2-(o- tolyl)ethyl]pyridine- 4-carboxamide | 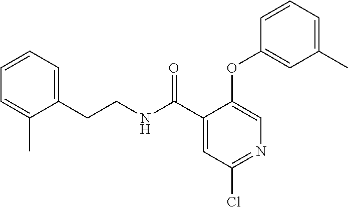 [SMILES: Cc1cccc(Oc2cnc(Cl)cc2C(=O)NCCc2ccccc2C)c1] | 1.86 | 380 | B |
| 1.57 | 2-chloro-5-(3- methylphenoxy)- N-[2-(p- tolyl)ethyl]pyridine- 4-carboxamide | 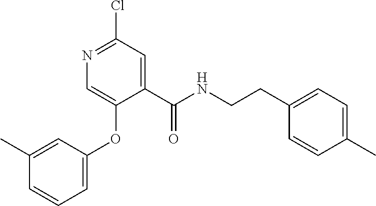 | 1.88 | 380 | B |
| 1.58 | 2-chloro-5-(3- cyclopropyl- phenoxy)-N-[2-(2,4- difluorophenyl)- 2-fluoro- ethyl]pyridine-4- carboxamide | 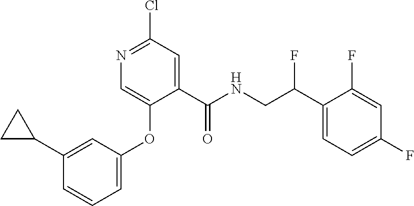 [SMILES: O=C(NCC(F)c1ccc(F)cc1F)c1cc(Cl)ncc1Oc1cccc(C2CC2)c1] | 1.87 | 446 | B |
| 1.59 | 2-chloro-N-[2- (2,4- difluorophenyl)- 2-fluoro-ethyl]-5- (3- methylphenoxy) pyridine-4- carboxamide | 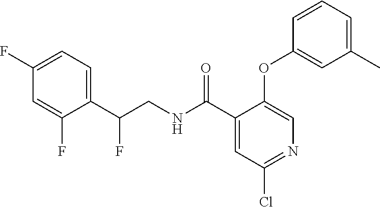 [SMILES: Cc1cccc(Oc2cnc(Cl)cc2C(=O)NCC(F)c2ccc(F)cc2F)c1] | 1.79 | 420 | B |
| 1.60 | N-[2-(2-chloro-4- methyl-phenyl)- 2-fluoro-ethyl]-5- (3- cyclopropyl- phenoxy)-2-methyl- pyridine-4-carboxamide | 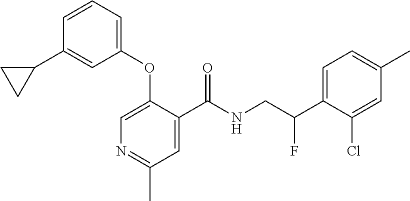 [SMILES: Cc1ccc(C(F)CNC(=O)c2cc(C)ncc2Oc2cccc(C3CC3)c2)c(Cl)c1] | 1.22 | 459 | A |
| 1.61 | 2-chloro-N-[2- (2,4- dichlorophenyl)- 2-fluoro-ethyl]-5- (3- methoxyphenoxy) pyridine-4- carboxamide | 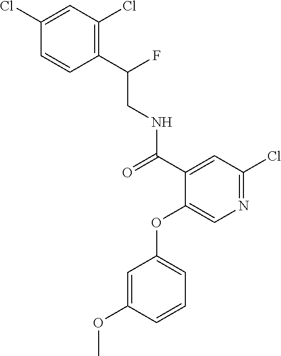 [SMILES: COc1cccc(Oc2cnc(Cl)cc2C(=O)NCC(F)c2ccc(Cl)cc2Cl)c1] | 2.02 | 468 | B |
| 1.62 | 2-chloro-N-[2- (2,4- dichlorophenyl)- 2-fluoro-ethyl]-5- (3,4- difluorophenoxy) pyridine-4- carboxamide | 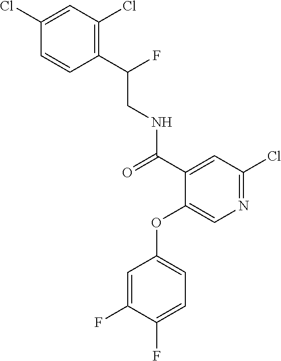 [SMILES: O=C(NCC(F)c1ccc(Cl)cc1Cl)c1cc(Cl)ncc1Oc1ccc(F)c(F)c1] | 2.00 | 474 | B |
| 1.63 | 2-chloro-5-(4- cyanophenoxy)- N-[2-(2,4- dichlorophenyl)- 2-fluoro- ethyl]pyridine-4- carboxamide | 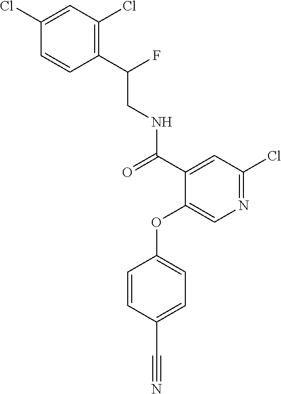 [SMILES: N#Cc1ccc(Oc2cnc(Cl)cc2C(=O)NCC(F)c2ccc(Cl)cc2Cl)cc1] | 1.87 | 463 | B |
| 1.64 | 2-chloro-5-(4- chlorophenoxy)- N-[2-(2,4- dichlorophenyl)- 2-fluoro- ethyl]pyridine-4- carboxamide | 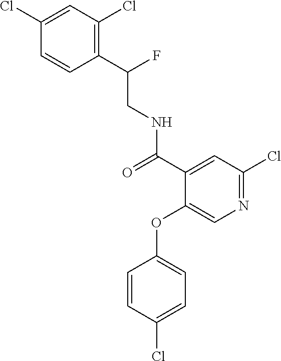 [SMILES: O=C(NCC(F)c1ccc(Cl)cc1Cl)c1cc(Cl)ncc1Oc1ccc(Cl)cc1] | 2.09 | 471 | B |
| 1.65 | 2-chloro-N-[2- (2,4- dichlorophenyl)- 2-fluoro-ethyl]-5- (3- ethoxyphenoxy) pyridine-4- carboxamide | 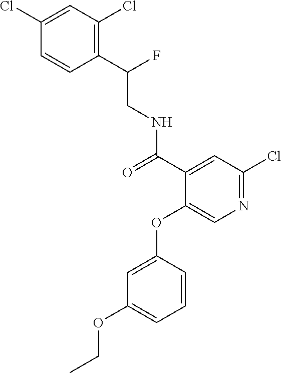 [SMILES: CCOc1cccc(Oc2cnc(Cl)cc2C(=O)NCC(F)c2ccc(Cl)cc2Cl)c1] | 2.12 | 482 | B |
| 1.66 | 2-chloro-N-[2- (2,4- dichlorophenyl)- 2-fluoro-ethyl]-5- (3- ethylphenoxy) pyridine-4- carboxamide | 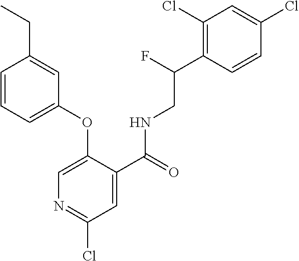 [SMILES: CCc1cccc(Oc2cnc(Cl)cc2C(=O)NCC(F)c2ccc(Cl)cc2Cl)c1] | 2.22 | 460 | B |
| 1.67 | 2-chloro-N-[2- (2,4- dichlorophenyl)- 2-fluoro-ethyl]-5- phenoxy- pyridine-4- carboxamide | 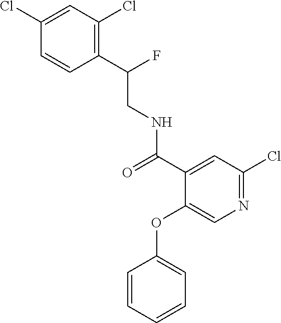 [SMILES: O=C(NCC(F)c1ccc(Cl)cc1Cl)c1cc(Cl)ncc1Oc1ccccc1] | 2.02 | 438 | B |
| 1.68 | 2-chloro-5-(3- chlorophenoxy)- N-[2-(2,4- dichlorophenyl) ethyl]pyridine- 4-carboxamide | 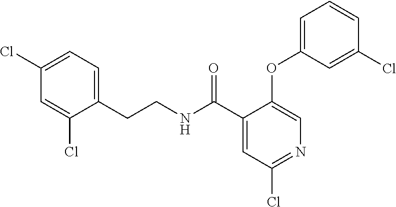 [SMILES: O=C(NCCc1ccc(Cl)cc1Cl)c1cc(Cl)ncc1Oc1cccc(Cl)c1] | 2.11 | 454 | B |
| 1.69 | 2-chloro-N-[2- (2,4- dichlorophenyl) ethyl]-5-(3- ethoxyphenoxy) pyridine-4- carboxamide | 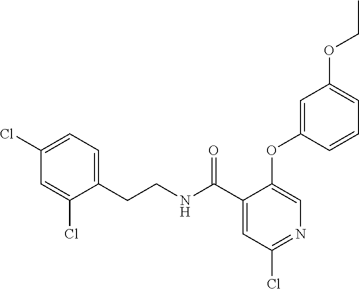 [SMILES: CCOc1cccc(Oc2cnc(Cl)cc2C(=O)NCCc2ccc(Cl)cc2Cl)c1] | 2.14 | 464 | B |
| 1.70 | 2-chloro-5-(3- cyclopropyl-2- fluoro-phenoxy)- N-[2-(2,4- dichlorophenyl)- 2-fluoro- ethyl]pyridine-4- carboxamide | 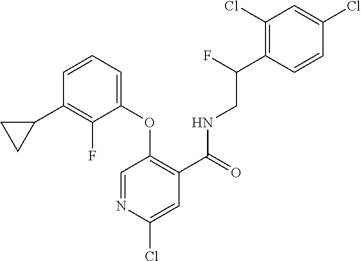 [SMILES: O=C(NCC(F)c1ccc(Cl)cc1Cl)c1cc(Cl)ncc1Oc1cccc(C2CC2)c1F] | 1.29 | 497 | A |
| 1.71 | 2-chloro-5-(3- cyclopropyl- phenoxy)-N-(2,2- difluoro-2- phenyl- ethyl)pyridine-4- carboxamide | 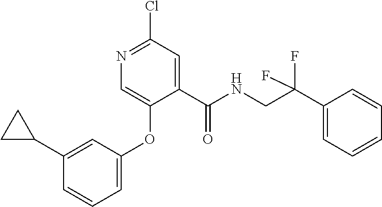 [SMILES: O=C(NCC(F)(F)c1ccccc1)c1cc(Cl)ncc1Oc1cccc(C2CC2)c1] | 1.88 | 428 | B |
| 1.72 | 2-chloro-N-[2-(4- cyanophenoxy) ethyl]-5-(3- cyclopropyl- phenoxy)pyridine- 4-carboxamide | 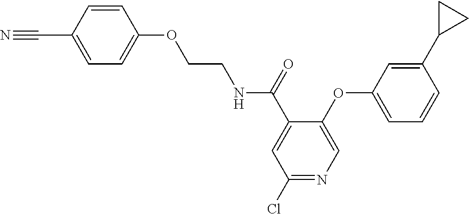 [SMILES: N#Cc1ccc(OCCNC(=O)c2cc(Cl)ncc2Oc2cccc(C3CC3)c2)cc1] | 1.69 | 433 | B |
| 1.73 | 2-chloro-N-[[1-(6- chloro-3- pyridyl)cyclo- propyl]methyl]-5-(3- cyclopropyl- phenoxy)pyridine-4- carboxamide | 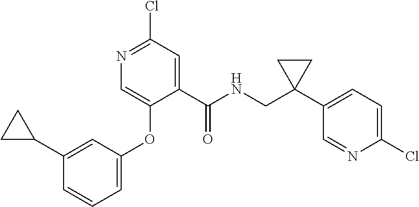 [SMILES: O=C(NCC1(c2ccc(Cl)nc2)CC1)c1cc(Cl)ncc1Oc1cccc(C2CC2)c1] | 1.80 | 453 | B |
| 1.74 | 2-chloro-N-[1-[(4- chlorophenyl) methyl]cyclopropyl]- 5-(3- cyclopropyl- phenoxy)pyridine- 4-carboxamide | 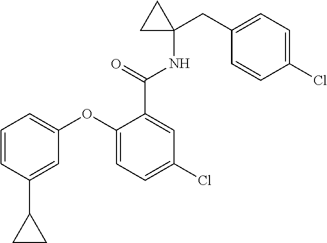 [SMILES: O=C(NC1(Cc2ccc(Cl)cc2)CC1)c1cc(Cl)ccc1Oc1cccc(C2CC2)c1] | 2.02 | 452 | B |
| 1.75 | 2-chloro-5-(3- cyclopropyl- phenoxy)-N-[(1R,2R)- 2-(4- fluorophenyl) cyclopropyl]pyridine- 4-carboxamide | 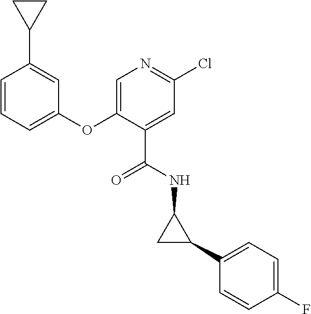 [SMILES: O=C(N[C@@H]1C[C@@H]1c1ccc(F)cc1)c1cc(Cl)ncc1Oc1cccc(C2CC2)c1] | 1.87 | 4.22 | B |
| 1.76 | 2-chloro-5-(3- cyclopropyl- phenoxy)-N-[2-(2,4- dimethylphenyl)- 2,2-difluoro- ethyl]pyridine- 4-carboxamide | 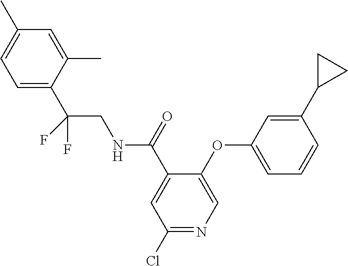 [SMILES: Cc1ccc(C(F)(F)CNC(=O)c2cc(Cl)ncc2Oc2cccc(C3CC3)c2)c(C)c1] | 2.06 | 456 | B |
| 1.77 | 2-chloro-5-(3- cyclopropyl- phenoxy)-N-[2-(2,4- dimethylphenyl) ethyl]pyridine-4- carboxamide | 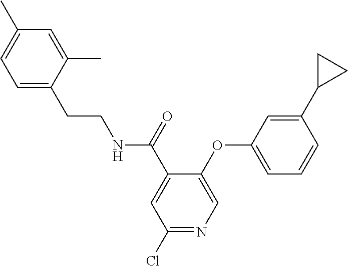 [SMILES: Cc1ccc(CCNC(=O)c2cc(Cl)ncc2Oc2cccc(C3CC3)c2)c(C)c1] | 2.05 | 420 | B |
| 1.78 | 2-chloro-5-(3- cyclopropyl- phenoxy)-N-[2-(2,4- dimethylphenyl)- 2-fluoro- ethyl]pyridine-4- carboxamide | 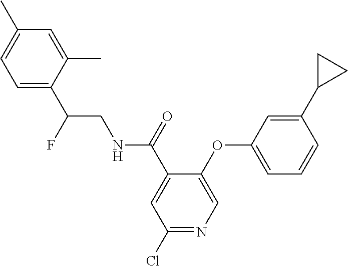 [SMILES: Cc1ccc(C(F)CNC(=O)c2cc(Cl)ncc2Oc2cccc(C3CC3)c2)c(C)c1] | 2.02 | 438 | B |
| 1.79 | 3-(3- cyclopropyl- phenoxy)-N-[2-(2,4- dichlorophenyl)- 2-fluoro-ethyl]-5- ethyl-pyridine-4- carboxamide | 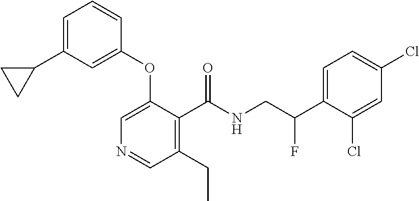 [SMILES: CCc1cncc(Oc2cccc(C3CC3)c2)c1C(=O)NCC(F)c1ccc(Cl)cc1Cl] | 1.19 | 473 | A |
| 1.80 | 2-chloro-5-(3- cyclopropyl- phenoxy)-N-[2-(4- ethyl-2-fluoro- phenyl)-2-fluoro- ethyl]pyridine-4- carboxamide | 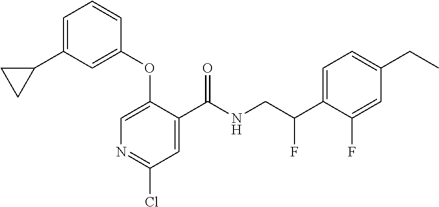 [SMILES: CCc1ccc(C(F)CNC(=O)c2cc(Cl)ncc2Oc2cccc(C3CC3)c2)c(F)c1] | 1.27 | 456 | A |
| 1.81 | 5-(3- cyclopropyl- phenoxy)-N-[2-(2,4- dichlorophenyl)- 2-fluoro-ethyl]-2- ethyl-pyridine-4- carboxamide | 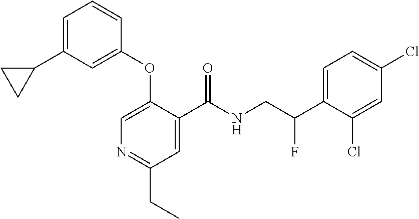 [SMILES: CCc1cc(C(=O)NCC(F)c2ccc(Cl)cc2Cl)c(Oc2cccc(C3CC3)c2)cn1] | 1.27 | 473 | A |
| 1.82 | 2-chloro-5-(3- cyclopropyl- phenoxy)-N-[2-(4- ethynyl-2-fluoro- phenyl)-2-fluoro- ethyl]pyridine-4- carboxamide | 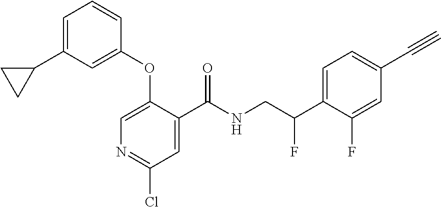 [SMILES: C#Cc1ccc(C(F)CNC(=O)c2cc(Cl)ncc2Oc2cccc(C3CC3)c2)c(F)c1] | 1.20 | 453 | A |
| 1.83 | 3-bromo-5-(3- cyclopropyl- phenoxy)-N-[2-(2,4- dichlorophenyl) ethyl]pyridine-4- carboxamide | 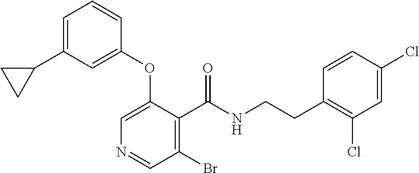 [SMILES: O=C(NCCc1ccc(Cl)cc1Cl)c1c(Br)cncc1Oc1cccc(C2CC2)c1] | 1.21 | 505, 507 | A |
| 1.84 | 3,5-bis(3- cyclopropyl- phenoxy)-N-[2-(2,4- dichlorophenyl)- 2-fluoro- ethyl]pyridine-4- carboxamide | 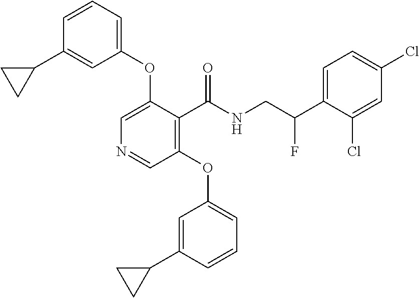 [SMILES: O=C(NCC(F)c1ccc(Cl)cc1Cl)c1c(Oc2cccc(C3CC3)c2)cncc1Oc1cccc(C2CC2)c1] | 1.21 | 525 | A |
| 1.85 | 3-(3- cyclopropyl- phenoxy)-N-[2-(2,4- dichlorophenyl)- 2-fluoro-ethyl]-5- (2- methylallyloxy) pyridine-4- carboxamide | 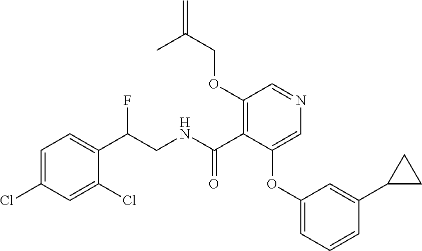 [SMILES: C=C(C)COc1cncc(Oc2cccc(C3CC3)c2)c1C(=O)NCC(F)c1ccc(Cl)cc1Cl] | 2.03 | 514 | B |
| 1.86 | 5-(3- cyclopropyl- phenoxy)-N-[2-(2,4- dichlorophenyl)- 2-fluoro-ethyl]-2- ethynyl-pyridine- 4-carboxamide | 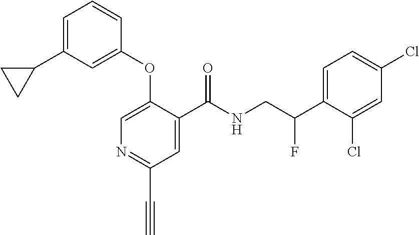 [SMILES: C#Cc1cc(C(=O)NCC(F)c2ccc(Cl)cc2Cl)c(Oc2cccc(C3CC3)c2)cn1] | 1.23 | 469 | A |
| 1.87 | 3-(3- cyclopropyl- phenoxy)-N-[2-(2,4- dichlorophenyl)- 2-fluoro-ethyl]-5- ethynyl-pyridine- 4-carboxamide | 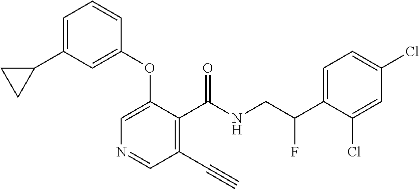 [SMILES: C#Cc1cncc(Oc2cccc(C3CC3)c2)c1C(=O)NCC(F)c1ccc(Cl)cc1Cl] | 1.17 | 469 | A |
| 1.88 | 3-(3- cyclopropyl- phenoxy)-N-[2-(2,4- dichlorophenyl) ethyl]-5-(1,1- dimethylprop-2- ynoxy)pyridine- 4-carboxamide | 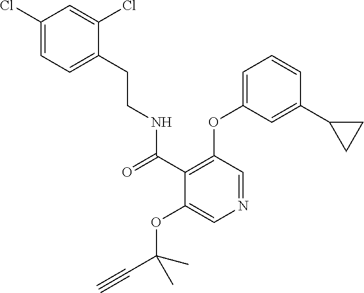 [SMILES: C#CC(C)(C)Oc1cncc(Oc2cccc(C3CC3)c2)c1C(=O)NCCc1ccc(Cl)cc1Cl] | 2.03 | 508 | B |
| 1.89 | 5-(3- cyclopropyl- phenoxy)-N-[2-(2,4- dichlorophenyl)- 2-fluoro-ethyl]-3- hydroxy-2-(2- methylprop-1- enyl)pyridine-4- carboxamide | 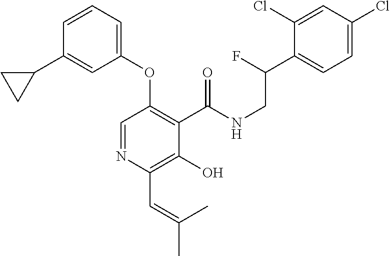 [SMILES: CC(C)=Cc1ncc(Oc2cccc(C3CC3)c2)c(C(=O)NCC(F)c2ccc(Cl)cc2Cl)c1O] | 1.45 | 515 | A |
| 1.90 | 5-(3- cyclopropyl- phenoxy)-N-[[1-(2,4- dichlorophenyl) cyclopropyl] methyl]-2-methyl- pyridine-4- carboxamide | 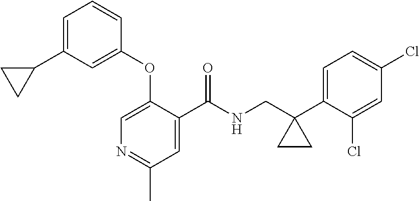 [SMILES: Cc1cc(C(=O)NCC2(c3ccc(Cl)cc3Cl)CC2)c(Oc2cccc(C3CC3)c2)cn1] | 1.69 | 467 | A |
| 1.91 | 3-(3- cyclopropyl- phenoxy)-N-[2-(2,4- dichlorophenyl)- 2-fluoro-ethyl]-5- methoxy- pyridine-4- carboxamide | 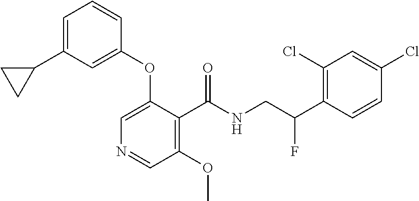 [SMILES: COc1cncc(Oc2cccc(C3CC3)c2)c1C(=O)NCC(F)c1ccc(Cl)cc1Cl] | 1.15 | 475 | A |
| 1.92 | 3-(3- cyclopropyl- phenoxy)-N-[2-(2,4- dichlorophenyl)- 2-fluoro-ethyl]-5- (1,1- dimethylprop-2- ynoxy)pyridine-4- carboxamide | 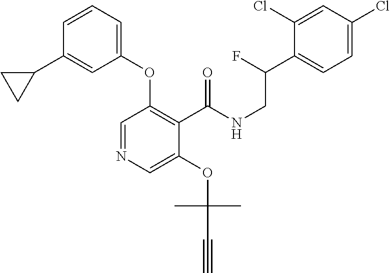 [SMILES: C#CC(C)(C)Oc1cncc(Oc2cccc(C3CC3)c2)c1C(=O)NCC(F)c1ccc(Cl)cc1Cl] | 1.29 | 527 | A |
| 1.93 | 3-(3- cyclopropyl- phenoxy)-N-[(2S)-2- (2,4- dichlorophenyl)- 2-fluoro-ethyl]-5- methyl-pyridine- 4-carboxamide | 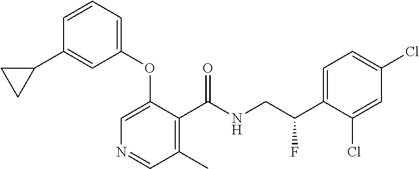 [SMILES: Cc1cncc(Oc2cccc(C3CC3)c2)c1C(=O)NC[C@@H](F)c1ccc(Cl)cc1Cl] | 1.15 | 459 | A |
| 1.94 | 3-(3- cyclopropyl- phenoxy)-N-[(2R)-2- (2,4- dichlorophenyl)- 2-fluoro-ethyl]-5- methyl-pyridine- 4-carboxamide | 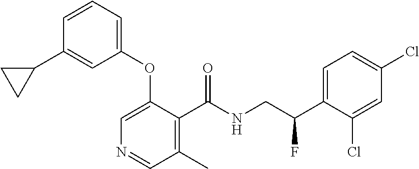 [SMILES: Cc1cncc(Oc2cccc(C3CC3)c2)c1C(=O)NC[C@H](F)c1ccc(Cl)cc1Cl] | 1.15 | 459 | A |
| 1.95 | 3-bromo-5-(3- cyclopropyl- phenoxy)-N-[(2R)-2- (2,4- dichlorophenyl)- 2-fluoro- ethyl]pyridine-4- carboxamide | 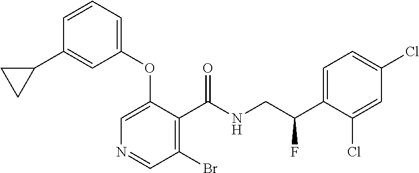 [SMILES: O=C(NC[C@H](F)c1ccc(Cl)cc1Cl)c1c(Br)cncc1Oc1cccc(C2CC2)c1] | 1.20 | 525, 523 | A |
| 1.96 | 3-(3- cyclopropyl- phenoxy)-N-[2-(2,4- dichlorophenyl)- 2-fluoro-ethyl]-5- hydroxy-pyridine- 4-carboxamide | 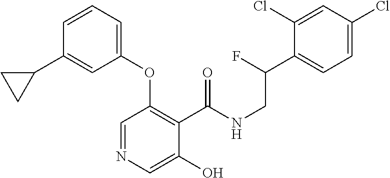 [SMILES: O=C(NCC(F)c1ccc(Cl)cc1Cl)c1c(O)cncc1Oc1cccc(C2CC2)c1] | 1.28 | 461 | A |
| 1.97 | 3-cyano-5-(3-cyclopropyl- phenoxy)-N-[2-(2,4- dichlorophenyl)- 2-fluoro- ethyl]pyridine-4- carboxamide | 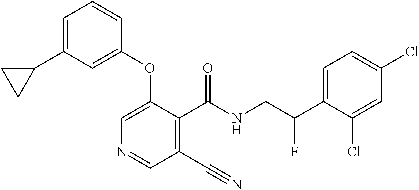 [SMILES: N#Cc1cncc(Oc2cccc(C3CC3)c2)c1C(=O)NCC(F)c1ccc(Cl)cc1Cl] | 1.99 | 470 | B |
| 1.98 | 5-(3- cyclopropyl- phenoxy)-N-[2-(2,4- dichlorophenyl)- 2-fluoro-ethyl]-2- methoxy- pyridine-4- carboxamide | 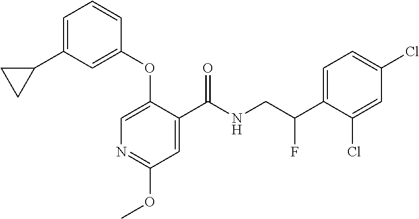 [SMILES: COc1cc(C(=O)NCC(F)c2ccc(Cl)cc2Cl)c(Oc2cccc(C3CC3)c2)cn1] | 1.28 | 474 | A |

### Biological Examples/Test Methods

### Alternaria solani (Early Blight of Tomato)

Tomato leaf disks are placed on water agar in multiwell plates (24-well format) and sprayed with test solutions. After drying, the leaf disks are inoculated with a spore suspension of the fungus. After appropriate incubation the activity of a compound is assessed 4 dpi (days post inoculation) as preventive fungicidal activity.

The following compounds from Table T1 gave at least 80% control of Alternaria solani at 200 ppm when compared to untreated control under the same conditions, which showed extensive disease development:
1.1, 1.7, 1.14, 1.20, 1.39, 1.41, 1.87, 1.94, 1.95, 1.98.

### Botryotinia fuckeliana syn. Botrytis cinerea (Gray Mould)

Conidia of the fungus from cryogenic storage were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 72 hrs.

The following compounds from Table T1 gave at least 80% control of Botryotinia fuckeliana at 20 ppm when compared to untreated control under the same conditions, which showed extensive disease development:
1.1, 1.2, 1.3, 1.4, 1.5, 1.6, 1.7, 1.8, 1.9,1.10, 1.13, 1.14, 1.15, 1.16, 1.17, 1.20, 1.21, 1.22, 1.24, 1.32, 1.35, 1.38, 1.39, 1.41, 1.42, 1.43, 1.45, 1.47, 1.53, 1.54, 1.55, 1.57, 1.58, 1.59, 1.60, 1.61, 1.62, 1.66, 1.67, 1.68, 1.70, 1.71, 1.75, 1.76, 1.77, 1.78, 1.79, 1.80, 1.81, 1.82, 1.84, 1.85, 1.86, 1.87, 1.88, 1.89, 1.90, 1.92, 1.91, 1.93, 1.94, 1.95, 1.96, 1.98.

### Botryotinia fuckeliana syn. Botrytis cinerea (Grey Mold on Tomato)

4-week old tomato plants cv. Roter Gnom are treated sprayed in a spray chamber with the formulated test compound diluted in water. The test plants are inoculated by spraying them with a spore suspension two days after application. The inoculated test plants are incubated at 20° C. and 95% rh in a greenhouse and the percentage leaf area covered by disease is assessed when an appropriate level of disease appears on untreated check plants (5-6 days after application).

The following compounds from Table T1 gave at least 50% control of Botryotinia fuckeliana at 200 ppm when compared to untreated control under the same conditions, which showed extensive disease development:
1.1, 1.3, 1.4, 1.5, 1.6, 1.7, 1.8, 1.9, 1.10, 1.11, 1.13, 1.14, 1.15, 1.16, 1.20, 1.21, 1.22, 1.35, 1.38, 1.39, 1.41, 1.43, 1.91, 1.60, 1.61, 1.70, 1.58, 1.70, 1.79, 1.80, 1.81, 1.82, 1.84, 1.85, 1.87, 1.88, 1.91.

### Glomerella lagenarium syn Colletotrichum lagenarium (Anthracnose)

Conidia of the fungus from cryogenic storage were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 72 hrs at 620 nm.

The following compounds from Table T1 gave at least 80% control of Glomerella lagenarium at 20 ppm when compared to untreated control under the same conditions, which showed extensive disease development:
1.1, 1.2, 1.3, 1.5, 1.6, 1.7, 1.8, 1.9, 1.10, 1.11, 1.12, 1.13, 1.14, 1.15, 1.16, 1.17, 1.19, 1.20, 1.21, 1.22, 1.23, 1.24, 1.26, 1.29, 1.31, 1.32, 1.33, 1.34, 1.35, 1.39, 1.41, 1.42, 1.43, 1.46, 1.47, 1.53, 1.57, 1.58, 1.60, 1.61, 1.63, 1.65, 1.66, 1.68, 1.71, 1.74, 1.75, 1.76, 1.77, 1.78, 1.79, 1.80, 1.81, 1.82, 1.84, 1.85, 1.86, 1.87, 1.88, 1.89, 1.90, 1,91, 1.92, 1.93, 1.94, 1.95, 1.96, 1.97, 1.98.

### Fusarium culmorum (Root Rot)

Conidia of the fungus from cryogenic storage were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 72 hrs.

The following compounds from Table T1 gave at least 80% control of Fusarium culmorum at 20 ppm when compared to untreated control under the same conditions, which showed extensive disease development:
1.1, 1.41, 1.67, 1.94.

### Phaeosphaeria nodorum syn. Septoria nodorum (Glume Blotch of Wheat)

Wheat leaf segments are placed on agar in multiwell plates (24-well format) and sprayed with test solutions. After drying, the leaf disks are inoculated with a spore suspension of the fungus. After appropriate incubation the activity of a compound is assessed 4 dpi (days after inoculation) as preventive fungicidal activity.

The following compounds from Table T1 gave at least 80% control of Phaeosphaeria nodorum at 200 ppm when compared to untreated control under the same conditions, which sowed extensive disease development:
1.1, 1.3, 1.7, 1.14, 1.39, 1.41, 1.86, 1.87, 1.92, 1.94, 1.95.

### Monographella nivalis syn. Microdochium nivale, Fusarium nivale (Snow Mould, Foot Rot)

Conidia of the fungus from cryogenic storage were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 72 hrs at 620 nm.

The following compounds from Table T1 gave at least 80% control of Monographella nivalis at 20 ppm when compared to untreated control under the same conditions, which showed extensive disease development:
1.1, 1.13, 1.14, 1.28, 1.36, 1.39, 1.41, 1.44, 1.46, 1.49, 1.50, 1.60, 1.63, 1.70, 1.78, 1.79, 1.81, 1.84, 1.85, 1.86, 1.87, 1.88, 1.92, 1.94, 1.95, 1.96.

### Pyrenophora teres (Net Blotch of Barley)

Barley leaf segments are placed on agar in multiwell plates (24-well format) and sprayed with test solutions. After drying, the leaf disks are inoculated with a spore suspension of the fungus. After appropriate incubation, the activity of a compound is assessed 4 dpi (days after inoculation) as preventive fungicidal activity.

The following compounds from Table T1 gave at least 80% control of Pyrenophora teres at 200 ppm when compared to untreated control under the same conditions, which showed extensive disease development:
1.1, 1.3, 1.14, 1.39, 1.41, 1.85, 1.87, 1.88, 1.94.

### Sclerotinia sclerotiorum (Cottony Rot, White Mold)

Mycelial fragments of the fungus prepared from a fresh liquid culture were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 72 hrs at 620 nm.

The following compounds from Table T1 gave at least 80% control of Sclerotinia sclerotiorum at 20 ppm when compared to untreated control under the same conditions, which showed extensive disease development:
1.1, 1.2, 1.3, 1.5, 1.7, 1.8, 1.13, 1.14, 1.20, 1.39, 1.41, 1.70, 1.79, 1.81, 1.85, 1.87, 1.88, 1.92, 1.94, 1.95, 1.96.

1. A compound of Formula (I): 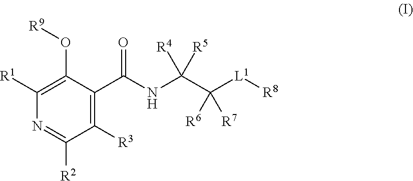
wherein:
L¹ is —O— or —CH₂— or a bond;
R¹ is selected from hydrogen, halogen, cyano, C₁-C₃-alkyl or C₁-C₃-alkoxy;
R² is selected from hydrogen, cyano, mercaptyl, halogen, C₁-C₃-alkyl, C₂-C₄-alkenyl, C₂-C₃-alkynyl, C₁-C₃-alkoxy, C₁-C₃-fluoroalkyl, C₁-C₃-fluoroalkoxy, C₃-C₄-cycloalkyl or C₁-C₃-alkyl sulfonyl;
R³ is selected from hydrogen, halogen, cyano, methyl, C₁-C₄-alkyl, C₁-C₃-fluoroalkyl, C₂-C₄-alkenyl, C₂-C₄-alkynyl, C₁-C₄-alkoxy, C₂-C₄-alkenyloxy, C₂-C₅-alkynyloxy, C₁-C₃-fluoroalkoxy, C₃-C₄-cycloalkyl; or
R³ is phenoxy optionally substituted with 1 or 2 substituents independently selected from halogen, cyano, methyl, ethyl, difluoromethyl, trifluoromethyl, methoxy, ethoxy, difluoromethoxy, trifluoromethoxy, cyclopropyl,
R⁴ and R⁵ are independently selected from hydrogen, cyano or methyl; or
R⁴ and R⁵, together with the carbon atom to which they are attached, form a cyclopropyl or cyclobutyl group;
R⁶ and R⁷ are independently selected from hydrogen, fluoro, chloro, methyl, trifluoromethyl, hydroxy, methoxy, methoxymethyl, difluoromethoxy, mercaptyl, methylsulfanyl; or
R⁶ and R⁷, together with the carbon atom to which they are attached, form an oxo, cyclopropyl, or cyclobutyl group, or a 4- to 6-membered monocyclic heterocycle group which comprises 1 or 2 heteroatoms individually selected from N, O and S; or
R⁴ and R⁶, together with the carbon atom to which they are attached, form a C₃-C₄-cycloalkyl or a 4- to 6-membered monocyclic heterocycle group which comprises 1 or 2 heteroatoms individually selected from N, O and S, and R⁵ and R⁷ are independently hydrogen or halogen;
R⁸ is phenyl optionally substituted with:
a single substituent selected from C₁-C₄-alkyl, C₁-C₂-haloalkyl, C₁-C₃-alkoxy, C₁-C₃-alkylsulfanyl, C₁-C₃-alkylsulfonyl, C₁-C₂-haloalkoxy, C₂-C₃-alkenyl, C₂-C₃-haloalkenyl, C₂-C₃-alkynyl, C₃-C₄-cycloalkyl, C₃-C₄-cycloalkyloxy or —NH(COC₁-C₃-alkyl), or
1, 2 or 3 substituents that may be the same or different, independently selected from hydroxyl, halogen, mercapto, amino, cyano, methyl, ethyl, propyl, isopropyl, difluoromethyl, trifluoromethyl, methoxy, ethoxy, propyloxy, isopropyloxy, methylsulfanyl, methylsulfonyl, difluoromethoxy, trifluoromethoxy, cyclopropyl, cyclobutyl, cyclopropyloxy, —NH(COCH₃); and
R⁹ is phenyl optionally substituted with:
a single substituent selected from C₁-C₄-alkyl, C₁-C₂-haloalkyl, C₁-C₄-alkoxy, C₁-C₃-alkylsulfanyl, C₁-C₃-alkylsulfonyl, C₁-C₂-haloalkoxy, C₂-C₃-alkenyl, C₂-C₃-haloalkenyl, C₂-C₃-alkynyl, C₃-C₄-cycloalkyl, C₃-C₄-cycloalkyloxy; or
1, 2 or 3 substituents which may be the same or different, independently selected from hydroxyl, halogen, mercapto, amino, cyano, methyl, ethyl, propyl, isopropyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, cyclopropyloxy, —NH(COCH₃); or
or a salt or an N-oxide thereof.

2. The compound according to claim 1, wherein L¹ is a bond.

3. The compound according to claim 1, wherein R¹ is hydrogen, chloro, methyl or methoxy, and preferably hydrogen.

4. The compound according to claim 1, wherein R² is is hydrogen, cyano, mercaptyl, halogen, methyl, methoxy, trifluoromethyl, cyclopropyl or methyl sulfonyl.

5. The compound according to claim 1, wherein R² is hydrogen, chloro or methyl.

6. The compound according to claim 1, wherein R³ is hydrogen, methyl, fluoro, chloro, bromo or cyano, methoxy.

7. The compound according to claim 1, wherein R⁴ and R⁵ are hydrogen.

8. The compound according to claim 1, wherein R⁶ is hydrogen or fluoro and R⁷ is hydrogen or fluoro.

9. The compound according to claim 1, wherein R⁸ is phenyl optionally substituted by 1 or 2 substituents independently selected from hydroxyl, halogen, cyano, methyl, ethyl, difluoromethyl, trifluoromethyl, methoxy or ethoxy.

10. The compound according to claim 1, wherein R⁹ is phenyl optionally substituted with 1 or 2 substituents independently selected from halogen, cyano, methyl, ethyl, difluoromethyl, trifluoromethyl, methoxy, ethoxy, propyloxy, iso-propyloxy, tert-butoxy, propynoxy, methylsulfanyl, difluoromethoxy, trifluoromethoxy, cyclopropyl and —NH(COCH₃).

11. The compound according to claim 1, wherein R⁹ is phenyl optionally substituted by a single substituent selected from ethyl, methyl, trifluoromethyl, cyclopropyl or cyano.

12. An agrochemical composition comprising a fungicidally effective amount of a compound according to claim 1.

13. The composition according to claim 12, further comprising at least one additional active ingredient and/or an agrochemically-acceptable diluent or carrier.

14. A method of controlling or preventing infestation of useful plants by phytopathogenic microorganisms, wherein a fungicidally effective amount of a compound according to claim 1, or a composition comprising this compound as active ingredient, is applied to the plants, to parts thereof or the locus thereof.

15. Use of a compound according to claim 1 as a fungicide.
